(19) United States
(12) Patent Application Publication
(10) Pub. No.: US 2021/0213091 A1
(43) Pub. Date: Jul. 15, 2021

(54) LIPOPHILIC ACTIVE AGENT INFUSED TOBACCO LEAVES AND/OR TOBACCO MATERIALS AND METHODS OF USE THEREOF

(71) Applicant: POVIVA CORP., Carson City, NV (US)

(72) Inventors: John Docherty, Port Perry (CA); Christopher Andrew Bunka, Kelowna (CA)

(73) Assignee: POVIVA CORP., Carson City, NV (US)

(21) Appl. No.: 17/215,600

(22) Filed: Mar. 29, 2021

Related U.S. Application Data
(63) Continuation-in-part of application No. 17/271,203, filed on Feb. 25, 2021.
(60) Provisional application No. 62/730,645, filed on Sep. 13, 2018.

(51) Int. Cl. A61K 36/81; A61K 31/4439; A61K 47/44; A61K 31/05; A61K 31/352; A61K 47/46; A61K 47/36; A24B 15/38; A24B 15/30; A24B 15/34; A24B 15/40

(52) U.S. Cl. CPC A61K 36/81; A61K 31/4439; A61K 47/44; A61K 31/05; A61K 31/352; A61K 47/46; A23F 3/14; A24B 15/385; A24B 15/303; A24B 15/34; A24B 15/302; A24B 15/406; A61K 47/36

(57) ABSTRACT

Aspects described herein relate to lipophilic active agent infused tobacco leaves and/or tobacco materials and methods of use thereof. More particularly, aspects described herein relate to tobacco leaves and/or tobacco materials infused with lipophilic active agents that provide enhanced bioavailability of the lipophilic active agents in a subject, and that mask unpleasant tastes.

### PRIORITY

This Application is a Continuation-In-Part Application of U.S. patent application Ser. No. 17/271,203, filed Feb. 25, 2021, which is a U.S. National Stage Application under 35 U.S.C. § 371, which claims the benefit of International Application PCT/US2019/051135, filed Sep. 13, 2019, which claims the benefit of U.S. Provisional Application No. 62/730,645, filed Sep. 13, 2018; which are incorporated herein by reference in their entirety.

### FIELD

Aspects described herein relate to lipophilic active agent infused tobacco leaves and/or tobacco materials and methods of use thereof. More particularly, aspects described herein relate to tobacco leaves and/or tobacco materials infused with lipophilic active agents that provide enhanced bioavailability of the lipophilic active agents in a subject, and that mask unpleasant tastes.

### BACKGROUND

Popular smoking articles, such as cigarettes, have a substantially cylindrical rod shaped structure and include a charge, roll or column of smokable material such as shredded tobacco (e.g., in cut filler form) surrounded by a paper wrapper thereby forming a so-called “tobacco rod.” Normally, a cigarette has a cylindrical filter element aligned in an end-to-end relationship with the tobacco rod. Typically, a filter element comprises plasticized cellulose acetate tow circumscribed by a paper material known as “plug wrap.” Certain cigarettes incorporate a filter element having multiple segments, and one of those segments can comprise activated charcoal particles. Typically, the filter element is attached to one end of the tobacco rod using a circumscribing wrapping material known as “tipping paper.” It also has become desirable to perforate the tipping material and plug wrap, in order to provide dilution of drawn mainstream smoke with ambient air. A cigarette is employed by a smoker by lighting one end thereof and burning the tobacco rod. The smoker then receives mainstream smoke into his/her mouth by drawing on the opposite end (e.g., the filter end) of the cigarette.

The tobacco used for cigarette manufacture is typically used in blended form. For example, certain popular tobacco blends, commonly referred to as “American blends,” comprise mixtures of flue-cured tobacco, burley tobacco and Oriental tobacco, and in many cases, certain processed tobaccos, such as reconstituted tobacco and processed tobacco stems. The precise amount of each type of tobacco within a tobacco blend used for the manufacture of a particular cigarette brand varies from brand to brand. However, for many tobacco blends, flue-cured tobacco makes up a relatively large proportion of the blend, while Oriental tobacco makes up a relatively small proportion of the blend. See, for example, Tobacco Encyclopedia, Voges (Ed.) p. 44-45 (1984), Browne, The Design of Cigarettes, 3^{rd} Ed., p. 43 (1990) and Tobacco Production, Chemistry and Technology, Davis et al. (Eds.) p. 346 (1999).

Tobacco also may be enjoyed in a so-called “smokeless” form. Particularly popular smokeless tobacco products are employed by inserting some form of processed tobacco or tobacco-containing formulation into the mouth of the user. Various types of smokeless tobacco products are set forth in U.S. Pat. No. 1,376,586 to Schwartz; U.S. Pat. No. 3,696,917 to Levi; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,987,907 to Townsend; U.S. Pat. No. 5,092,352 to Sprinkle, III et al.; and U.S. Pat. No. 5,387,416 to White et al.; U.S. Pat. Appl. Pub. No. 2005/0244521 to Strickland et al.; PCT WO 04/095959 to Arnarp et al.; PCT WO 05/063060 to Atchley et al.; PCT WO 05/004480 to Engstrom; PCT WO 05/016036 to Bjorkholm; and PCT WO 05/041699 to Quinter et al., each of which is incorporated herein by reference. See, for example, the types of smokeless tobacco formulations, ingredients, and processing methodologies set forth in U.S. Pat. No. 6,953,040 to Atchley et al. and U.S. Pat. No. 7,032,601 to Atchley et al., each of which is incorporated herein by reference.

One type of smokeless tobacco product is referred to as “snuff.” Representative types of moist snuff products, commonly referred to as “snus,” are manufactured in Europe, particularly in Sweden, by or through companies such as Swedish Match AB, Fiedler & Lundgren AB, Gustavus AB, Skandinavisk Tobakskompagni A/S, and Rocker Production AB. Snus products available in the U.S.A. are marketed under the tradenames Camel Snus Frost, Camel Snus Original and Camel Snus Spice by R. J. Reynolds Tobacco Company. Representative smokeless tobacco products also are marketed under the tradenames Oliver Twist by House of Oliver Twist A/S; Copenhagen, Skoal, SkoalDry, Rooster, Red Seal, Husky, and Revel by U.S. Smokeless Tobacco Co.; “taboka” by Philip Morris USA; and Levi Garrett, Peachy, Taylor's Pride, Kodiak, Hawken Wintergreen, Grizzly, Dental, Kentucky King, and Mammoth Cave by Conwood Sales Co., L.P. See also, for example, Bryzgalov et al., 1N1800 Life Cycle Assessment, Comparative Life Cycle Assessment of General Loose and Portion Snus (2005). In addition, certain quality standards associated with snus manufacture have been assembled as a so-called GothiaTek standard.

Through the years, various treatment methods and additives have been proposed for altering the overall character or nature of tobacco materials utilized in tobacco compositions. For example, additives or treatment processes are sometimes utilized in order to alter the chemistry or sensory properties of the tobacco material, or in the case of smokable tobacco materials, to alter the chemistry or sensory properties of mainstream smoke generated by smoking articles including the tobacco material. In some cases, a heat treatment process can be used to impart a desired color or visual character to the tobacco material, desired sensory properties to the tobacco material, or a desired physical nature or texture to the tobacco material.

In particular, the sensory attributes of cigarette smoke can be enhanced by incorporating flavoring materials into various components of a cigarette. See, Leffingwell et al., Tobacco Flavoring for Smoking Products, R. J. Reynolds Tobacco Company (1972). Exemplary flavoring additives include menthol and products of Maillard reactions, such as pyrazines, aminosugars, and Amadori compounds. Various processes for preparing flavorful and aromatic compositions for use in tobacco compositions are set forth in U.S. Pat. No. 3,424,171 to Rooker; U.S. Pat. No. 3,476,118 to Luttich; U.S. Pat. No. 4,150,677 to Osborne, Jr. et al.; U.S. Pat. No. 4,986,286 to Roberts et al.; U.S. Pat. No. 5,074,319 to White et al.; U.S. Pat. No. 5,099,862 to White et al.; U.S. Pat. No. 5,235,992 to Sensabaugh, Jr.; U.S. Pat. No. 6,298,858 to Coleman, III et al.; U.S. Pat. No. 6,325,860 to Coleman, III et al.; U.S. Pat. No. 6,428,624 to Coleman, III et al.; U.S. Pat. No. 6,440,223 to Dube et al.; U.S. Pat. No. 6,499,489 to Coleman, III; and U.S. Pat. No. 6,591,841 to White et al.; U.S. Pat. Appl. Publication No. 2004/0173228 to Coleman, III; and U.S. application Ser. No. 12/191,751 to Coleman, III et al., filed Aug. 14, 2008, each of which is incorporated herein by reference. Such processes often include the application of heat to a tobacco material, which can result in reactions that form certain byproducts.

The sensory attributes of smokeless tobacco can also be enhanced by incorporation of certain flavoring materials. See, for example, U.S. Pat. Appl. Pub. Nos. 2002/0162562 to Williams; 2002/0162563 to Willams; 2003/0070687 to Atchley et al.; 2004/0020503 to Williams, 2005/0178398 to Breslin et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0029117 to Mua et al.; 2008/0173317 to Robinson et al.; and 2008/0209586 to Neilsen et al., each of which is incorporated herein by reference.

Accordingly, it would be desirable in the art to provide further methods for altering the character and nature of tobacco (and tobacco compositions and formulations) useful in smoking articles or smokeless tobacco products, including enhancement of bioavailability of active agents, masking of unpleasant tastes, and the incorporation of additional active agents.

### SUMMARY

To address the foregoing problems, in whole or in part, and/or other problems that may have been observed by persons skilled in the art, the present disclosure provides compositions and methods as described by way of example as set forth below.

In one aspect, lipophilic active agent infused tobacco leaves and/or tobacco materials are provided, comprising:

(a) a therapeutically effective amount of a lipophilic active agent, wherein the lipophilic active agent is selected from the group consisting of cannabinoids, terpenes and terpenoids, NSAIDs, vitamins, nicotine compounds, phosphodiesterase type 5 (PDE5) inhibitors, Maca extract, estrogen, progestin, testosterone, buprenorphine, and scopolamine;

(b) a bioavailability enhancing agent, wherein the bioavailability enhancing agent enhances the bioavailability of the lipophilic active agent in a subject; and

(c) tobacco leaves and/or tobacco materials.

In other aspects, the lipophilic active agent infused tobacco leaves and/or tobacco materials are obtainable by the steps of:

(i) contacting the tobacco leaves and/or tobacco materials with an oil comprising the lipophilic active agent and the bioavailability enhancing agent; and

(ii) dehydrating the tobacco leaves and/or tobacco materials;

thereby producing the lipophilic active agent infused tobacco leaves and/or tobacco materials. In further aspects, step (i) comprises saturating the tobacco leaves and/or tobacco materials in the oil comprising the lipophilic active agent and the bioavailability enhancing agent. In further aspects, the bioavailability enhancing agent is an edible oil comprising long chain fatty acids, medium chain fatty acids, and/or a combination of both medium and long chain fatty acids.

Tobacco compositions comprising any of the lipophilic active agent infused tobacco leaves and/or tobacco materials described herein are also provided.

Smokeless tobacco compositions comprising any of the lipophilic active agent infused tobacco leaves and/or tobacco materials described herein are also provided.

In another aspect, a process is provided for making lipophilic active agent infused tobacco leaves and/or tobacco materials comprising the steps of:

(a) contacting tobacco leaves and/or tobacco materials with an oil comprising a lipophilic active agent and a bioavailability enhancing agent; and

(b) dehydrating the tobacco leaves and/or tobacco materials;

thereby producing lipophilic active agent infused tobacco leaves and/or tobacco materials; wherein the lipophilic active agent infused tobacco leaves and/or tobacco materials comprise a therapeutically effective amount of the lipophilic active agent, and further wherein:

(i) the lipophilic active agent is selected from the group consisting of cannabinoids, terpenes and terpenoids, NSAIDs, vitamins, nicotine compounds, phosphodiesterase type 5 (PDE5) inhibitors, Maca extract, estrogen, progestin, testosterone, buprenorphine, and scopolamine and

(ii) the bioavailability enhancing agent enhances the bioavailability of the lipophilic active agent.

In other aspects, step (a) comprises saturating the food product in the oil comprising the lipophilic active agent and the bioavailability enhancing agent. In further aspects, the bioavailability enhancing agent is an edible oil comprising long chain fatty acids, medium chain fatty acids, and/or a combination of both medium and long chain fatty acids. In further aspects, step (a) further comprises contacting the tobacco leaves and/or tobacco materials with a flavoring agent, particularly wherein the flavoring agent is selected from the group consisting of vanilla, vanillin, ethyl vanillin, orange oil, peppermint oil, strawberry, raspberry, and mixtures thereof.

Tobacco compositions comprising any of the lipophilic active agent infused tobacco leaves and/or tobacco materials made by any of the processes described herein are also provided.

Smokeless tobacco compositions comprising any of the lipophilic active agent infused tobacco leaves and/or tobacco materials made by any of the processes described herein are also provided.

Other compositions, methods, features, and advantages of the disclosure will be or will become apparent to one with skill in the art upon examination of the following detailed description. It is intended that all such additional compositions, methods, features, and advantages be included within this description, be within the scope of the disclosure, and be protected by the accompanying claims.

The nicotine infused tobacco leaves cam be prepared as follows:

(i) contacting the tobacco leaves with a source of nicotine and an edible oil to form a nicotine composite; and

(ii) dehydrating the composite of tobacco leaves and/or tobacco materials; thereby producing the lipophilic active agent infused tobacco leaves and/or tobacco materials. In further aspects, step (i) comprises saturating the tobacco leaves and/or tobacco materials in the oil comprising the lipophilic active agent and the bioavailability enhancing agent. In further aspects, the bioavailability enhancing agent is an edible oil comprising long chain fatty acids, medium chain fatty acids, and/or a combination of both medium and long chain fatty acids.

### DETAILED DESCRIPTION

The present disclosure is directed to lipophilic active agent infused tobacco leaves and/or tobacco materials and methods for making lipophilic active agent infused tobacco leaves and/or tobacco materials. More particularly, aspects described herein relate to lipophilic active agent infused tobacco leaves and/or tobacco materials that provide enhanced bioavailability of the lipophilic active agents in a subject, and/or that mask unpleasant tastes of lipophilic active agents and/or tobacco.

The lipophilic active agent infused tobacco leaves and/or tobacco materials of the disclosure can be used as an additive for a smoking article (e.g., as part of the smokable blend or as an additive to the filter or wrapping paper of the smoking article) or as a smokeless tobacco composition, such as loose moist snuff, loose dry snuff, chewing tobacco, pelletized tobacco pieces, extruded or formed tobacco strips, pieces, rods, or sticks, finely divided ground powders, finely divided or milled agglomerates of powdered pieces and components, flake-like pieces, molded processed tobacco pieces, pieces of tobacco-containing gum, rolls of tape-like films, readily water dissolvable or water-dispersible formats such as films, strips, or powders, shelf-stable ready to drink beverage compositions, and pharmaceutical compositions formulated in unit dosage form, including but not limited to tablets, caplets, capsules, lozenges, films, strips, gelcaps, and syrups.

Such pharmaceutical compositions may also comprise one or more pharmaceutically acceptable excipient(s), which are pharmacologically inactive components such as a diluent, disintegrant, carrier, or the like. Additional information concerning pharmaceutical formulations is found in Remington (2006) The Science and Practice of Pharmacy, 21^{st} edition, Lippincott Williams & Wilkins.

The lipophilic active agent infusion process of the disclosure can also be incorporated into conventional tobacco treatment processes, such as processes adapted to faun flavorful and aromatic compounds (e.g., Maillard reaction products), processes adapted for pasteurization of tobacco compositions, processes for preparing tobacco casing products, reconstituted tobacco processes (e.g., cast sheet and paper-making reconstituted tobacco processes), tobacco extraction processes, reordering processes, toasting processes, steam treatments, and drying processes.

Accordingly, the lipophilic active agent infused tobacco leaves and/or tobacco materials of the disclosure can be used within exemplary technologies for conventional and smokeless tobacco compositions and processes described in the published U.S. Patent Applications listed Appendix D, each of which are incorporated herein in their entireties.

### COMPOSITIONS

### Lipophilic Active Agent Infused Tobacco Leaves and/or Tobacco Materials

In one aspect, lipophilic active agent infused tobacco leaves and/or tobacco materials are provided, comprising:
(a) a therapeutically effective amount of a lipophilic active agent, wherein the lipophilic active agent is selected from the group consisting of cannabinoids, terpenes and terpenoids, NSAIDs, vitamins, nicotine compounds, phosphodiesterase type 5 (PDE5) inhibitors, Maca extract, estrogen, progestin, testosterone, buprenorphine, and scopolamine
(b) a bioavailability enhancing agent, wherein the bioavailability enhancing agent enhances the bioavailability of the lipophilic active agent in a subject; and
(c) tobacco leaves and/or tobacco materials.

In another aspect, the bioavailability enhancing agent comprises an edible oil comprising long chain fatty acids. In another aspect, the bioavailability enhancing agent comprises an edible oil comprising a medium chain fatty acid. In further aspects, the bioavailability enhancing agent is a combination of edible oils that include both medium and long chain fatty acids.

In another aspect, the lipophilic active agent infused tobacco leaves and/or tobacco materials are obtainable by the steps of:
(i) contacting the tobacco leaves and/or tobacco materials with an oil comprising the lipophilic active agent and the bioavailability enhancing agent; and
(ii) dehydrating the tobacco leaves and/or tobacco materials; thereby producing the lipophilic active agent infused tobacco leaves and/or tobacco materials.

In a further aspect, step (i) comprises saturating the tobacco leaves and/or tobacco materials in the oil comprising the lipophilic active agent and the bioavailability enhancing agent. In yet another aspect, the lipophilic active agent infused tobacco leaves and/or tobacco materials further comprise a flavoring agent. In a further aspect, the lipophilic active agent infused food tobacco leaves and/or tobacco materials are lyophilized.

As will be evident from the present disclosure, the bioavailability agent is either an edible oil or fat, or a protective colloid as defined herein.

In general, the following aspects relate to the following nicotine infused tobacco leaves, comprising:
(a) a therapeutically effective amount of nicotine or a synthetic compound that is capable of delivering nicotine, especially in use;
(b) a bioavailability enhancing agent; and
(c) tobacco leaves.

The infused tobacco leaves can further comprise other optional adjunct ingredients as further disclosed herein. Non-limiting examples of which include flavorants, fillers, binders, pH adjusters, buffering agents, colorants, disintegration aids, antioxidants, humectants, and preservatives.

Compounds which are “capable of delivering include” include complexes wherein nicotine is bound to a polymeric material. Non-limiting examples include polyhmethacrylic acid ion-exchange resin, for example, Amberlite IRP64, Purolite C115HMR or Doshion P551. Other examples include nicotine that is microencapsulated or enrobed in a biodegradable, food grade polymer.

The disclosed nicotine infused tobacco leaves, comprise:
(a) from about 0.01% to about 1% by weight of nicotine;
(b) from about 0.05% to about 5% by weight of a bioavailability enhancing agent; and
(c) the balance tobacco leaves.

Another aspect of the present disclosure relates to nicotine infused tobacco leaves, comprising:
(a) a therapeutically effective amount of nicotine;
(b) an edible oil; and
(c) tobacco leaves.

For the purposes of the present disclosure the above composition is also known herein as a “composite.” Contacting a source of nicotine and an edible oil with tobacco leaves prior to dehydration results in a “composite.” The subsequently dehydrated composite yields a disclose composition. As disclosed herein below, the composite, as well as, the final composition can comprise other materials.

As used herein the term “DehydraTECH™” can refer to the final nicotine infused tobacco leaves or by way of reference to the process for preparing all iterations of the disclosed nicotine infused tobacco leaves. In addition, terms such as “pre-DehydraTECH™” and the like refer to intermediate steps in preparing the disclosed nicotine infused tobacco leaves as intermediates prior to the final processing step, i.e., dehydration.

A non-limiting embodiment of this aspect of the disclosed nicotine infused tobacco leaves comprise:
(a) a therapeutically effective amount of nicotine;
(b) an edible oil selected from the group consisting of borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, soybean oil, sunflower oil, tree nut oil, hydrogenated castor oil, hydrogenated coconut oil, hydrogenated palm oil, hydrogenated soybean oil, hydrogenated vegetable oil, glyceryl trioleate, glyceryl trilinoleate, glyceryl trilinolenate, citrate thisocetyl triglyceride having 10-18 carbon atoms, omega-3 polyunsaturated fatty acid triglyceride containing oil, omega-3 oil, omega-6 oil, and any combination thereof, and
(c) tobacco leaves;
wherein the nicotine infused tobacco leaves are dehydrated.

In another embodiment the edible oil is selected from the group consisting of canola oil, coconut oil, corn oil, cottonseed oil, olive oil, palm oil, palm kernel oil, rapeseed oil, safflower oil, soybean oil, sunflower oil, hydrogenated coconut oil, hydrogenated palm oil, hydrogenated soybean oil, and any combination thereof.

Non-limiting examples of compositions include infused tobacco leaves wherein the bioavailability agent is sunflower oil. Non-limiting examples of compositions include infused tobacco leaves wherein the bioavailability agent is sunflower oil.

The example above, for example, can be prepared by the DehydraTECH™ process which comprises dehydrating either the final composition or intermediate compositions, wherein the intermediate compositions can be combined with one or more disclosed ingredients which then requires a further dehydration step.

In one iteration of this aspect, nicotine infused tobacco leaves, comprise:
(a) a therapeutically effective amount of nicotine;
(b) an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil; and
(c) tobacco leaves.

In a further aspect nicotine infused tobacco leaves, comprise:
(a) a therapeutically effective amount of nicotine;
(b) an edible oil;
(c) a binder; and
(d) tobacco leaves.

In one iteration of this aspect the nicotine infused tobacco leaves, comprise:
(a) a therapeutically effective amount of nicotine;
(b) an edible oil;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In a further iteration of this aspect the nicotine infused tobacco leaves, comprise:
(a) a therapeutically effective amount of nicotine;
(b) an edible oil an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In one embodiment of this iteration of this aspect, the nicotine infused tobacco leaves, comprise:
(a) a therapeutically effective amount of nicotine;
(b) sunflower oil;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In another embodiment of this iteration of this aspect, the nicotine infused tobacco leaves, comprise:
(a) a therapeutically effective amount of nicotine;
(b) an edible oil an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil;
(c) gum arabic; and
(d) tobacco leaves.

A non-limiting example of this embodiment comprises:
(a) a therapeutically effective amount of nicotine;
(b) sunflower oil;
(c) gum arabic; and
(d) tobacco leaves

A still further aspect of the present disclosure relates to nicotine infused tobacco leaves, comprising:
(a) a therapeutically effective amount of nicotine;
(b) a protective colloid chosen from whole milk, skimmed milk, non-fat dry milk or mixtures thereof; and
(c) tobacco leaves.

A non-limiting embodiment of this aspect of the disclosed nicotine infused tobacco leaves comprise:
(a) a therapeutically effective amount of nicotine;
(b) non-fat dry milk; and
(c) tobacco leaves;

wherein the final nicotine infused tobacco leaves are dehydrated.

The example above, for example, can be prepared by the herein above disclosed DehydraTECH™ process which comprises dehydrating either the final composition or intermediate compositions, wherein the intermediate compositions can be combined with one or more disclosed ingredients which then requires a further dehydration step.

In a further aspect nicotine infused tobacco leaves, comprise:
(a) a therapeutically effective amount of nicotine;
(b) a protective colloid;
(c) a binder; and
(d) tobacco leaves.

In one iteration of this aspect the nicotine infused tobacco leaves, comprise:
(a) a therapeutically effective amount of nicotine;
(b) a protective colloid;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In a further iteration of this aspect the nicotine infused tobacco leaves, comprise:
(a) a therapeutically effective amount of nicotine;
(b) a protective colloid chosen from whole milk, skimmed milk, non-fat dry milk or mixtures thereof;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In one embodiment of this iteration of this aspect, the nicotine infused tobacco leaves, comprise:
(a) a therapeutically effective amount of nicotine;
(b) non-fat dry milk;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In another embodiment of this iteration of this aspect, the nicotine infused tobacco leaves, comprise:
(a) a therapeutically effective amount of nicotine;
(b) a protective colloid chosen from whole milk, skimmed milk, non-fat dry milk or mixtures thereof;
(c) gum arabic; and
(d) tobacco leaves.

A non-limiting example of this embodiment comprises:
(a) a therapeutically effective amount of nicotine;
(b) non-fat dry milk;
(c) gum arabic; and
(d) tobacco leaves

A yet still further aspect of the present disclosure relates to nicotine infused tobacco leaves, comprising:
(a) a therapeutically effective amount of nicotine;
(b) a bioavailability enhancing agent chosen from an edible oil or a protective colloid;
(c) one or more cannabinoids; and
(d) tobacco leaves.

A non-limiting embodiment of this aspect of the disclosed nicotine infused tobacco leaves comprise:
(a) a therapeutically effective amount of nicotine;
(b) an edible oil selected from the group consisting of borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, soybean oil, sunflower oil, tree nut oil, hydrogenated castor oil, hydrogenated coconut oil, hydrogenated palm oil, hydrogenated soybean oil, hydrogenated vegetable oil, glyceryl trioleate, glyceryl trilinoleate, glyceryl trilinolenate, citrate thisocetyl triglyceride having 10-18 carbon atoms, omega-3 polyunsaturated fatty acid triglyceride containing oil, omega-3 oil, omega-6 oil, and any combination thereof,
(c) one or more cannabinoids; and
(d) tobacco leaves;
wherein the nicotine infused tobacco leaves are dehydrated.

In one iteration of this aspect, the nicotine infused tobacco leaves, comprise:
(a) a therapeutically effective amount of nicotine;
(b) an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil;
(c) one or more cannabinoids selected from the group consisting of tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C₄, cannabidivarinic acid, cannabidiorcol, Δ⁹-tetrahydrocannabinolic acid A, delta-9-tetrahydrocannabinolic acid B, Δ⁹-tetrahydrocannabinolic acid-C₄, Δ⁹-tetrahydrocannabi-varinic acid, Δ⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C₂, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9-propyl-2,6-methano-2H-1-benzoxocin-5-methanol-cannabiripsol, trihydroxy-Δ⁹-tetrahydrocannabinol, and cannabinol; and
(d) tobacco leaves.

In a further iteration of this aspect, the nicotine infused tobacco leaves, comprise:
(a) a therapeutically effective amount of nicotine;
(b) an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil;
(c) one or more cannabinoids selected from the group consisting of tetrahydrocannabinol, cannabidiol, and mixtures thereof; and
(d) tobacco leaves.

A non-limiting example of this iteration of this aspect of the nicotine infused tobacco leaves, comprises:
(a) a therapeutically effective amount of nicotine;
(b) sunflower oil;
(c) one or more cannabinoids selected from the group consisting of tetrahydrocannabinol, cannabidiol, and mixtures thereof; and
(d) tobacco leaves.

A further non-limiting example of this iteration of this aspect of the nicotine infused tobacco leaves, comprises:
(a) a therapeutically effective amount of nicotine;
(b) sunflower oil;
(c) tetrahydrocannabinol; and
(d) tobacco leaves.

Another non-limiting example of this iteration of this aspect of the nicotine infused tobacco leaves, comprises:
(a) a therapeutically effective amount of nicotine;
(b) sunflower oil;
(c) cannabidiol; and
(d) tobacco leaves.

A still yet further aspect of the disclosed nicotine infused tobacco leaves comprises:
(a) a therapeutically effective amount of nicotine;
(b) a protective colloid;
(c) one or more cannabinoids; and
(d) tobacco leaves;
wherein the nicotine infused tobacco leaves are dehydrated.

In one embodiment of this aspect, the nicotine infused tobacco leaves comprise:
(a) a therapeutically effective amount of nicotine;
(b) a protective colloid chosen from whole milk, skimmed milk, non-fat dry milk or mixtures thereof;
(c) one or more cannabinoids selected from the group consisting of tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C₄, cannabidivarinic acid, cannabidiorcol, Δ⁹-tetrahydrocannabinolic acid A, delta-9-tetrahydrocannabinolic acid B, Δ⁹-tetrahydrocannabinolic acid-C₄, Δ⁹-tetrahydrocannabi-varinic acid, Δ⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C₂, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9-propyl-2,6-methano-2H-1-benzoxocin-5-methanol-cannabiripsol, trihydroxy-Δ⁹-tetrahydrocannabinol, and cannabinol; and
(d) tobacco leaves.

In a further embodiment of this aspect the nicotine infused tobacco leaves, comprise:
(a) a therapeutically effective amount of nicotine;
(b) a protective colloid chosen from whole milk, skimmed milk, non-fat dry milk or mixtures thereof;
(c) one or more cannabinoids selected from the group consisting of tetrahydrocannabinol, cannabidiol, and mixtures thereof; and
(d) tobacco leaves.

A non-limiting iteration this aspect of the nicotine infused tobacco leaves, comprises:
(a) a therapeutically effective amount of nicotine;
(b) non-fat dry milk;
(c) one or more cannabinoids selected from the group consisting of tetrahydrocannabinol, cannabidiol, and mixtures thereof; and
(d) tobacco leaves.

A non-limiting example of this iteration of this aspect of the nicotine infused tobacco leaves, comprises:
(a) a therapeutically effective amount of nicotine;
(b) non-fat dry milk;
(c) tetrahydrocannabinol; and
(d) tobacco leaves.

Another non-limiting example of this iteration of this aspect of the nicotine infused tobacco leaves, comprises:
(a) a therapeutically effective amount of nicotine;
(b) non-fat dry milk;
(c) cannabidiol; and
(d) tobacco leaves.

A further aspect of the nicotine infused tobacco leaves, comprise:
(a) a therapeutically effective amount of nicotine;
(b) a protective colloid;
(c) a cannabinoid;
(d) a binder; and
(e) tobacco leaves.

In one embodiment of this aspect the nicotine infused tobacco leaves, comprise:
(a) a therapeutically effective amount of nicotine;
(b) a protective colloid;
(c) a cannabinoid;
(d) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(e) tobacco leaves.

In a further embodiment of this aspect the nicotine infused tobacco leaves, comprise:
(a) a therapeutically effective amount of nicotine;
(b) a protective colloid chosen from whole milk, skimmed milk, non-fat dry milk or mixtures thereof;
(c) one or more cannabinoids selected from the group consisting of tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C₄, cannabidivarinic acid, cannabidiorcol, Δ⁹-tetrahydrocannabinolic acid A, delta-9-tetrahydrocannabinolic acid B, Δ⁹-tetrahydrocannabinolic acid-C₄, Δ⁹-tetrahydrocannabi-varinic acid, Δ⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C₂, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9-propyl-2,6-methano-2H-1-benzoxocin-5-methanol-cannabiripsol, trihydroxy-Δ⁹-tetrahydrocannabinol, and cannabinol;
(d) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(e) tobacco leaves.

In one iteration of this embodiment of this aspect, the nicotine infused tobacco leaves, comprise:
(a) a therapeutically effective amount of nicotine;
(b) non-fat dry milk;
(c) one or more cannabinoids;
(d) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(e) tobacco leaves.

A non-limiting example of this iteration of this embodiment of this aspect of the nicotine infused tobacco leaves, comprises:
(a) a therapeutically effective amount of nicotine;
(b) non-fat dry milk;
(c) tetrahydrocannabinol;
(d) gum arabic; and
(e) tobacco leaves.

A further non-limiting example of this iteration of this embodiment of this aspect of the nicotine infused tobacco leaves, comprises:
(a) a therapeutically effective amount of nicotine;
(b) non-fat dry milk;
(c) cannabidiol;
(d) gum arabic; and
(e) tobacco leaves.

A still yet further aspect of the present disclosure relates to the use of nicotine composites to deliver increased nicotine to tobacco leaves. The term “nicotine composite” as used herein refers to nicotine or compound that can equally stand for nicotine that is bound to or otherwise combined with an inert carrier. Non-limiting examples of nicotine composites include nicotine bound to an ion-exchange resin, inter alia, (polymethacrylic acid, such as Amberlite™ IRP64™, Purolite™ C115HMR™ or Doshion™ P551™ Non-limiting examples include nicotine polacrilex and nicotine resinate.

As such, a still yet further aspect of the disclosure relates to a nicotine composite infused tobacco leaves comprises:
(a) a therapeutically effective amount of nicotine bound to polymethacrylic acid;
(b) a protective colloid;
(c) one or more cannabinoids; and
(d) tobacco leaves;
wherein the nicotine composite infused tobacco leaves are dehydrated.

In one embodiment of this aspect, the nicotine composite infused tobacco leaves comprise:
(a) a therapeutically effective amount of nicotine bound to polymethacrylic acid;
(b) a protective colloid chosen from whole milk, skimmed milk, non-fat dry milk or mixtures thereof;
(c) one or more cannabinoids selected from the group consisting of tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C₄, cannabidivarinic acid, cannabidiorcol, Δ⁹-tetrahydrocannabinolic acid A, delta-9-tetrahydrocannabinolic acid B, Δ⁹-tetrahydrocannabinolic acid-C₄, Δ⁹-tetrahydrocannabi-varinic acid, Δ⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C₂, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9-propyl-2,6-methano-2H-1-benzoxocin-5-methanol-cannabiripsol, trihydroxy-Δ⁹-tetrahydrocannabinol, and cannabinol; and
(d) tobacco leaves.

In a further embodiment of this aspect the nicotine composite infused tobacco leaves, comprise:
(a) a therapeutically effective amount of nicotine bound to polymethacrylic acid;
(b) a protective colloid chosen from whole milk, skimmed milk, non-fat dry milk or mixtures thereof;
(c) one or more cannabinoids selected from the group consisting of tetrahydrocannabinol, cannabidiol, and mixtures thereof; and
(d) tobacco leaves.

A non-limiting iteration this aspect of the nicotine composite infused tobacco leaves, comprises:
(a) a therapeutically effective amount of nicotine bound to a polymer;
(b) non-fat dry milk;
(c) one or more cannabinoids selected from the group consisting of tetrahydrocannabinol, cannabidiol, and mixtures thereof; and
(d) tobacco leaves.

A non-limiting example of this iteration of this aspect of the nicotine composite infused tobacco leaves, comprises:
(a) a therapeutically effective amount of nicotine bound to a polymer;
(b) non-fat dry milk;
(c) tetrahydrocannabinol; and
(d) tobacco leaves.

Another non-limiting example of this iteration of this aspect of the nicotine composite infused tobacco leaves, comprises:
(a) a therapeutically effective amount of nicotine bound to polymethacrylic acid;
(b) non-fat dry milk;
(c) cannabidiol; and
(d) tobacco leaves.

A further aspect of the nicotine composite infused tobacco leaves, comprise:
(a) a therapeutically effective amount of nicotine bound to polymethacrylic acid;
(b) a protective colloid;
(c) a cannabinoid;
(d) a binder; and
(e) tobacco leaves.

In one embodiment of this aspect the nicotine composite infused tobacco leaves, comprise:
(a) a therapeutically effective amount of nicotine bound to polymethacrylic acid;
(b) a protective colloid;
(c) a cannabinoid;
(d) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(e) tobacco leaves.

In a further embodiment of this aspect the nicotine composite infused tobacco leaves, comprise:
(a) a therapeutically effective amount of nicotine bound to polymethacrylic acid;
(b) a protective colloid chosen from whole milk, skimmed milk, non-fat dry milk or mixtures thereof;
(c) one or more cannabinoids selected from the group consisting of tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C₄, cannabidivarinic acid, cannabidiorcol, Δ⁹-tetrahydrocannabinolic acid A, delta-9-tetrahydrocannabinolic acid B, Δ⁹-tetrahydrocannabinolic acid-C₄, Δ⁹-tetrahydrocannabi-varinic acid, Δ⁹-tetrahydrocannabivarin, Δ⁹-tetrahydrocannabiorcolic acid, Δ⁹-tetrahydro-cannabiorcol, Δ⁷-cis-iso-tetrahydrocannabivarin, Δ⁸-tetrahydrocannabiniolic acid, Δ⁸-tetrahydrocannabinol, cannabicyclolic acid, cannabicylovarin, cannabielsoic acid A, cannabielsoic acid B, cannabinolic acid, cannabinol methylether, cannabinol-C₄, cannabinol-C₂, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, Δ⁹-cis-tetrahydrocannabinol, 3,4,5,6-tetrahydro-7-hydroxy-α,α-2-trimethyl-9-propyl-2,6-methano-2H-1-benzoxocin-5-methanol-cannabiripsol, trihydroxy-Δ⁹-tetrahydrocannabinol, and cannabinol;
(d) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(e) tobacco leaves.

In one iteration of this embodiment of this aspect, the nicotine composite infused tobacco leaves, comprise:
(a) a therapeutically effective amount of nicotine bound to polymethacrylic acid;
(b) non-fat dry milk;
(c) one or more cannabinoids;
(d) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(e) tobacco leaves.

A non-limiting example of this iteration of this embodiment of this aspect of the nicotine composite infused tobacco leaves, comprises:
(a) a therapeutically effective amount of nicotine bound to polymethacrylic acid;
(b) non-fat dry milk;
(c) tetrahydrocannabinol;
(d) gum arabic; and
(e) tobacco leaves.

A further non-limiting example of this iteration of this embodiment of this aspect of the nicotine composite infused tobacco leaves, comprises:
(a) a therapeutically effective amount of nicotine bound to polymethacrylic acid;
(b) non-fat dry milk;
(c) cannabidiol;
(d) gum arabic; and
(e) tobacco leaves.

All of the above aspects, iteration, embodiments and examples depicted herein above are dehydrated after being combined. The ingredients can be combined in any order, for example, the nicotine and edible oil can be admixed, followed by combination with other ingredients, infusion onto the tobacco leaves followed by dehydration.

The compositions can be further processed by grinding, shredding, chopping and the like to form flowable solids that can be administered to a subject.

Tobacco leaves and/or tobacco materials as used in the compositions and methods of the present disclosure may vary. The tobacco leaves and/or tobacco materials may include types of tobaccos such as flue-cured tobacco, burley tobacco, sun-cured tobacco (e.g., Oriental tobacco or Indian Kurnool), Maryland tobacco, dark tobacco, dark-fired tobacco, dark air cured (e.g., passanda, cubano, jatin and bezuki tobaccos) or light air cured (e.g., North Wisconsin and galpoa tobaccos), and Rustica tobaccos, as well as other rare or specialty tobaccos or even green or uncured tobaccos.

Descriptions of various types of tobaccos, growing practices, harvesting practices and curing practices are set forth in Tobacco Production, Chemistry and Technology, Davis et al. (Eds.) (1999), which is incorporated herein by reference. See, also, the types of tobaccos that are set forth in U.S. Pat. No. 4,660,577 to Sensabaugh, Jr. et al.; U.S. Pat. No. 5,387,416 to White et al.; and U.S. Pat. No. 6,730,832 to Dominguez et al., each of which is incorporated herein by reference.

Most preferably, the tobacco materials are those that have been appropriately cured and aged. Especially preferred techniques and conditions for curing flue-cured tobacco are set forth in Nestor et al., Beitrage Tabakforsch. Int., 20 (2003) 467-475 and U.S. Pat. No. 6,895,974 to Peele, which are incorporated herein by reference. Representative techniques and conditions for air curing tobacco are set forth in Roton et al., Beitrage Tabakforsch. Int., 21 (2005) 305-320 and Staaf et al., Beitrage Tabakforsch. Int., 21 (2005) 321-330, which are incorporated herein by reference.

Certain types of unusual or rare tobaccos can be sun cured. Manners and methods for improving the smoking quality of Oriental tobaccos are set forth in U.S. Pat. No. 7,025,066 to Lawson et al., which is incorporated herein by reference. Representative Oriental tobaccos include katerini, prelip, komotini, xanthi and yambol tobaccos. Tobacco compositions including dark air cured tobacco are set forth in US Patent Appl. Pub. No. 2008/0245377 to Marshall et al., which is incorporated herein by reference.

Lipophilic active agent infused tobacco leaves and/or tobacco materials of the present disclosure, such as lipophilic active agent infused tobacco leaves and/or tobacco materials intended to be used in a smokeless form, may incorporate a single type of tobacco (e.g., in a so-called “straight grade” form). For example, the tobacco within the compositions and methods of the present disclosure may be composed solely of flue-cured tobacco (e.g., all of the tobacco may be composed, or derived from, either flue-cured tobacco lamina or a mixture of flue-cured tobacco lamina and flue-cured tobacco stem). The tobacco within the compositions and methods of the present disclosure also may have a so-called “blended” form. For example, the tobacco within the compositions and methods of the present disclosure may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other exemplary tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other exemplary tobacco blends incorporate about 20 to about 30 parts Oriental tobacco and about 70 to about 80 parts flue-cured tobacco.

As used herein, “tobacco leaves and/or tobacco materials” includes whole or partial leaves of tobacco, processed tobacco parts or pieces, cured and aged tobacco in essentially natural lamina or stem form, a tobacco extract, extracted tobacco pulp (e.g., using water as a solvent), or a mixture of the foregoing (e.g., a mixture that combines extracted tobacco pulp with granulated cured and aged natural tobacco lamina). The tobacco leaves and/or tobacco materials most preferably includes tobacco lamina, or tobacco lamina and stem mixture. Tobacco mixtures incorporating a predominant amount of tobacco lamina, relative to tobacco stem, are preferred. Most preferably, the tobacco lamina and stem are used in an unextracted form, that is, such that the extractable portion (e.g., the water soluble portion) is present within the unextractable portion (e.g., the tobacco pulp) in a manner comparable to that of natural tobacco provided in a cured and aged form. Portions of the tobacco may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Patent Appl. Pub. No. 2004/0182404 to Poindexter, et al., all of which are incorporated by reference. In addition, the tobacco leaves and/or tobacco materials optionally may incorporate tobacco that has been fermented. See, also, the types of tobacco processing techniques set forth in PCT WO 05/063060 to Atchley et al., which is incorporated herein by reference.

The tobacco leaves and/or tobacco materials within the compositions and methods of the present disclosure are typically provided in a shredded, ground, granulated, fine particulate, or powder form. Most preferably, the tobacco leaves and/or tobacco materials is employed in the form of parts or pieces that have an average particle size less than that of the parts or pieces of shredded tobacco used in so-called “fine cut” tobacco products. Typically, the very finely divided tobacco particles or pieces are sized to pass through a screen of about 18 Tyler mesh, generally are sized to pass a screen of about 20 Tyler mesh, often are sized to pass through a screen of about 50 Tyler mesh, frequently are sized to pass through a screen of about 60 Tyler mesh, may even be sized to pass through a screen of 100 Tyler mesh, and further may be sized so as to pass through a screen of 200 Tyler mesh. If desired, air classification equipment may be used to ensure that small sized tobacco particles of the desired sizes, or range of sizes, may be collected. In one embodiment, the tobacco material is in particulate form sized to pass through an 18 Tyler mesh, but not through a 60 Tyler mesh. If desired, differently sized pieces of granulated tobacco may be mixed together. Typically, the very finely divided tobacco particles or pieces suitable for snus products have a particle size greater than −8 Tyler mesh, often −8 to +100 Tyler mesh, frequently −18 to +60 Tyler mesh.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent.

Tobacco extracts are useful as components of the tobacco leaves and/or tobacco materials within the compositions and methods of the present disclosure. Extracts can be used in solid form (e.g., spray-dried or freeze-dried form), in liquid form, in semi-solid form, or the like. Exemplary tobacco extracts and extraction techniques are set forth, for example, in U.S. Pat. No. 4,150,677 to Osborne, Jr. et al.; U.S. Pat. No. 4,967,771 to Fagg et al.; U.S. Pat. No. 5,005,593 to Fagg et al.; U.S. Pat. No. 5,148,819 to Fagg; and U.S. Pat. No. 5,435,325 to Clapp et al., all of which are incorporated by reference herein. Various tobacco extraction and reconstitution methodologies are set forth in U.S. Pat. No. 5,065,775 to Fagg; U.S. Pat. No. 5,360,022 to Newton; and U.S. Pat. No. 5,131,414 to Fagg, all of which are incorporated by reference herein. See also, the tobacco extract treatment methodologies set forth in U.S. Pat. No. 5,131,415 to Munoz et al. and U.S. Pat. No. 5,318,050 to Gonzalez-Parra, both of which are incorporated by reference herein.

Suitable known reconstituted tobacco processing techniques, such as paper-making techniques or casting-type processes, can be employed in conjunction with the processes of the disclosure. See, for example, the types of paper-making processes set forth in U.S. Pat. No. 3,398,754 to Tughan; U.S. Pat. No. 3,847,164 to Mattina; U.S. Pat. No. 4,131,117 to Kite; U.S. Pat. No. 4,270,552 to Jenkins; U.S. Pat. No. 4,308,877 to Mattina; U.S. Pat. No. 4,341,228 to Keritsis; U.S. Pat. No. 4,421,126 to Gellatly; U.S. Pat. No. 4,706,692 to Gellatly; U.S. Pat. No. 4,962,774 to Thomasson; U.S. Pat. No. 4,941,484 to Clapp; U.S. Pat. No. 4,987,906 to Young; U.S. Pat. No. 5,056,537 to Brown; U.S. Pat. No. 5,143,097 to Sohn; U.S. Pat. No. 5,159,942 to Brinkley et al.; U.S. Pat. No. 5,325,877 to Young; U.S. Pat. No. 5,445,169 to Brinkley; U.S. Pat. No. 5,501,237 to Young; U.S. Pat. No. 5,533,530 to Young; which are incorporated herein by reference. See, for example, the casting processes set forth in U.S. Pat. No. 3,353,541 to Hind; U.S. Pat. No. 3,399,454 to Hind; U.S. Pat. No. 3,483,874 to Hind; U.S. Pat. No. 3,760,815 to Deszyck; U.S. Pat. No. 4,674,519 to Keritsis; U.S. Pat. No. 4,972,854 to Kiernan; U.S. Pat. No. 5,023,354 to Hickle; U.S. Pat. No. 5,099,864 to Young; U.S. Pat. No. 5,101,839 to Jakob; U.S. Pat. No. 5,203,354 to Hickle; U.S. Pat. No. 5,327,917 to Lekwauwa; U.S. Pat. No. 5,339,838 to Young; U.S. Pat. No. 5,598,868 to Jakob; U.S. Pat. No. 5,715,844 to Young; U.S. Pat. No. 5,724,998 to Gellatly; and U.S. Pat. No. 6,216,706 to Kumar; and EPO 565360; EPO 1055375 and PCT WO 98/01233; which are incorporated herein by reference. Extracts, extracted materials, and slurries used in traditional types of reconstituted tobacco processes can be employed as ingredients in tobacco formulations of the disclosure.

The processes of the disclosure can be used in connection with any tobacco treatment process where the application of heat is involved, and in conjunction with heat treatment processing aids or additives or in conjunction with ingredients such as casing components. See, for example, the casing materials and methods set forth in U.S. Pat. No. 4,177,822 to Bryant, Jr. et al.; U.S. Pat. No. 4,306,577 to Wu et al.; U.S. Pat. No. 4,449,541 to Mays et al.; U.S. Pat. No. 4,537,204 to Gaisch et al.; U.S. Pat. No. 4,819,668 to Shelar et al.; and U.S. Pat. No. 4,836,224 to Lawson et al., each of which is incorporated by reference herein.

### Tobacco Compositions Comprising Lipophilic Active Agent Infused Tobacco Leaves and/or Tobacco Materials

The lipophilic active agent infused tobacco leaves and/or tobacco materials of the present disclosure are useful as additives for the manufacture of smoking articles (also referenced herein as “tobacco compositions”). For example, lipophilic active agent infused tobacco leaves and/or tobacco materials prepared in accordance with the present disclosure can be mixed with casing materials and applied to tobacco as a casing ingredient, incorporated into smoking articles as a top dressing ingredient, or incorporated into reconstituted tobacco materials. Still further, the lipophilic active agent infused tobacco leaves and/or tobacco materials of the disclosure can be incorporated into a cigarette filter (e.g., in the filter plug, plug wrap, or tipping paper) or incorporated into cigarette wrapping paper, preferably on the inside surface, during the cigarette manufacturing process. The lipophilic active agent infused tobacco leaves and/or tobacco materials can also be used as an additive within certain aerosol-generating electronic smoking articles, such as those described in U.S. Pat. Appl. Pub. No. 2008/0092912 to Robinson et al., which is incorporated by reference herein in its entirety.

The lipophilic active agent infused tobacco leaves and/or tobacco materials of the disclosure may also be incorporated into the tobacco blends, representative cigarette components, and representative cigarettes manufactured therefrom, set forth in U.S. Pat. No. 4,836,224 to Lawson et al.; U.S. Pat. No. 4,924,888 to Perfetti et al.; U.S. Pat. No. 5,056,537 to Brown et al.; U.S. Pat. No. 5,220,930 to Gentry; and U.S. Pat. No. 5,360,023 to Blakley et al.; US Pat. Application 2002/0000235 to Shafer et al.; and PCT WO 02/37990. Those tobacco materials also can be employed for the manufacture of those types of cigarettes that are described in U.S. Pat. No. 4,793,365 to Sensabaugh; U.S. Pat. No. 4,917,128 to Clearman et al.; U.S. Pat. No. 4,947,974 to Brooks et al.; U.S. Pat. No. 4,961,438 to Korte; U.S. Pat. No. 4,920,990 to Lawrence et al.; U.S. Pat. No. 5,033,483 to Clearman et al.; U.S. Pat. No. 5,074,321 to Gentry et al.; U.S. Pat. No. 5,105,835 to Drewett et al.; U.S. Pat. No. 5,178,167 to Riggs et al.; U.S. Pat. No. 5,183,062 to Clearman et al.; U.S. Pat. No. 5,211,684 to Shannon et al.; U.S. Pat. No. 5,247,949 to Deevi et al.; U.S. Pat. No. 5,551,451 to Riggs et al.; U.S. Pat. No. 5,285,798 to Banerjee et al.; U.S. Pat. No. 5,593,792 to Farrier et al.; U.S. Pat. No. 5,595,577 to Bensalem et al.; U.S. Pat. No. 5,816,263 to Counts et al.; U.S. Pat. No. 5,819,751 to Barnes et al.; U.S. Pat. No. 6,095,153 to Beven et al.; U.S. Pat. No. 6,311,694 to Nichols et al.; and U.S. Pat. No. 6,367,481 to Nichols, et al.; and PCT WO 97/48294 and PCT WO 98/16125. See, also, those types of commercially marketed cigarettes described Chemical and Biological Studies on New Cigarette Prototypes that Heat Instead of Burn Tobacco, R. J. Reynolds Tobacco Company Monograph (1988) and Inhalation Toxicology, 12:5, p. 1-58 (2000).

The lipophilic active agent infused tobacco leaves and/or tobacco materials of the disclosure can also be used as a smokeless tobacco product or incorporated as an additive in a smokeless tobacco product. Various types of smokeless tobacco products are set forth in U.S. Pat. No. 1,376,586 to Schwartz; U.S. Pat. No. 3,696,917 to Levi; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,987,907 to Townsend; U.S. Pat. No. 5,092,352 to Sprinkle, III et al.; and U.S. Pat. No. 5,387,416 to White et al.; US Pat. App. Pub. No. 2005/0244521 to Strickland et al.; PCT WO 04/095959 to Arnarp et al.; PCT WO 05/063060 to Atchley et al.; PCT WO 05/004480 to Engstrom; PCT WO 05/016036 to Bjorkholm; and PCT WO 05/041699 to Quinter et al., each of which is incorporated herein by reference. See also, the types of smokeless tobacco formulations, ingredients, and processing methodologies set forth in U.S. Pat. No. 6,953,040 to Atchley et al. and U.S. Pat. No. 7,032,601 to Atchley et al.; U.S Pat. Appl. Pub. Nos. 2002/0162562 to Williams; 2002/0162563 to Willams; 2003/0070687 to Atchley et al.; 2004/0020503 to Williams, 2005/0178398 to Breslin et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0029117 to Mua et al.; 2008/0173317 to Robinson et al.; and 2008/0209586 to Neilsen et al., each of which is incorporated herein by reference.

The relative amount of lipophilic active agent infused tobacco leaves and/or tobacco materials within tobacco formulations may vary. Preferably, the amount of lipophilic active agent infused tobacco leaves and/or tobacco materials within the tobacco formulation is at least about 10 percent or at least about 25 percent, on a dry weight basis of the formulation. In certain instances, the amounts of other components within the tobacco formulation may exceed about 40 percent, on a dry weight basis. A typical range of tobacco material within the formulation is about 10 to about 60 weight percent, more often about 20 to about 40 weight percent on a dry basis.

Tobacco products differ uniquely from food products with regard to certain reactions, such as a reaction between asparagine and reducing sugars. With smoking tobacco products (e.g., cigarettes, cigars, pipe tobacco), the temperature gradient during use is much higher than the temperature encountered in foods during cooking, which can lead to an increased rate of reaction. With certain smokeless tobacco products, the pH can be much higher than the pH of foods and, during processing, heating the tobacco with an increased pH may enhance the rate of certain reactions. Therefore, inhibition of certain reactions can be particularly challenging when dealing with tobacco products.

Accordingly, exemplary additives to tobacco formulations that include lipophilic active agent infused tobacco leaves and/or tobacco materials of the present disclosure include amino acids, compositions incorporating di- and trivalent cations, asparaginase, certain non-reducing saccharides, certain reducing agents, phenolic compounds (e.g., compounds having at least one phenolic functionality), certain compounds having at least one free thiol group or functionality, oxidizing agents, oxidation catalysts, rosemary extract (or other plant extracts derived from herbal or botanical sources), and combinations thereof. Without being bound of a theory of operation, it is believed that these additives are capable of inhibiting reaction of asparagine to form acrylamide, either by providing competing reactions that preferentially react with available reducing sugars, by chemical interaction with asparagine that renders it unable to react with reducing sugars, by chemical interaction with reaction intermediates, or by chemical interaction with acrylamide. Use of certain additives according to the disclosure is described in U.S. Pat. No. 7,037,540 to Elder et al. and U.S. Pat. No. 7,267,834 to Elder et al.; and U.S. Pat. Appl. Pub. Nos. 2004/0058046 to Zyzak et al; 2005/0196504 to Finley; 2006/0194743 to Oku et al; 2007/0141225 to Elder et al.; 2007/0141227 to Boudreaux et al.; and 2007/0166439 to Soe et al., which are incorporated by reference in their entirety.

The amount of the additive present in tobacco formulations that include lipophilic active agent infused tobacco leaves and/or tobacco materials of the present disclosure will vary depending on the desired character of the tobacco formulation and the type of additive selected. Typically, the amount of additive is at least about 0.01 dry weight percent, more often at least about 0.1 dry weight percent, and most often at least about 1 dry weight percent. The additive is present in an amount typically less than about 15 dry weight percent, such as less than about 10 weight percent or less than about 8 weight percent. In one embodiment, the amount of the additive is about 1 dry weight percent to about 5 dry weight percent.

In some embodiments, the tobacco formulations that include lipophilic active agent infused tobacco leaves and/or tobacco materials of the present disclosure are smokeless tobacco compositions. Such smokeless tobacco compositions, in addition to tobacco, water, and additives as noted elsewhere herein, also typically include additional components such as flavorants, fillers, binders, pH adjusters, buffering agents, colorants, disintegration aids, antioxidants, humectants, and preservatives.

Exemplary flavorants that can be used are components, or suitable combinations of those components, that act to alter the bitterness, sweetness, sourness, or saltiness of the smokeless tobacco product, enhance the perceived dryness or moistness of the formulation, or the degree of tobacco taste exhibited by the formulation. Types of flavorants include salts (e.g., sodium chloride, potassium chloride, sodium citrate, potassium citrate, sodium acetate, potassium acetate, and the like), natural sweeteners (e.g., fructose, sucrose, glucose, maltose, mannose, galactose, lactose, and the like), artificial sweeteners (e.g., sucralose, saccharin, aspartame, acesulfame K, neotame, and the like); and mixtures thereof. The amount of flavorants utilized in the tobacco composition can vary, but is typically up to about 10 dry weight percent, and certain embodiments are characterized by a flavorant content of at least about 1 dry weight percent, such as about 1 to about 10 dry weight percent. Combinations of flavorants are often used, such as about 0.1 to about 2 dry weight percent of an artificial sweetener and about 0.5 to about 8 dry weight percent of a salt such as sodium chloride.

Exemplary filler materials include vegetable fiber materials such as sugar beet fiber materials (e.g., FIBREX® brand filler available from International Fiber Corporation), oats or other cereal grain (including processed or puffed grains), bran fibers, starch, or other modified or natural cellulosic materials such as microcrystalline cellulose. Additional specific examples include corn starch, maltodextrin, dextrose, calcium carbonate, calcium phosphate, lactose, manitol, xylitol, and sorbitol. The amount of filler utilized in the tobacco composition can vary, but is typically up to about 50 dry weight percent, and certain embodiments are characterized by a filler content of at least about 10 dry weight percent, such as about 20 to about 50 dry weight percent. Combinations of fillers are often used, such as about 2 to about 8 dry weight percent of calcium carbonate, about 10 to about 20 dry weight percent of rice flour, and about 10 to about 20 weight percent of maltodextrin.

Typical binders include povidone, sodium carboxymethylcellulose and other modified cellulosic materials, sodium alginate, xanthan gum, starch-based binders, gum arabic, pectin, carrageenan, pullulan, zein, and the like. The amount of binder utilized in the tobacco composition can vary, but is typically up to about 30 dry weight percent, and certain embodiments are characterized by a binder content of at least about 5 dry weight percent, such as about 5 to about 30 dry weight percent.

Preferred pH adjusters or buffering agents provide and/or buffer within a pH range of about 6 to about 10, and exemplary agents include metal hydroxides, metal carbonates, metal bicarbonates, and mixtures thereof Specific exemplary materials include sodium hydroxide, potassium hydroxide, potassium carbonate, sodium carbonate, and sodium bicarbonate. The amount of pH adjuster or buffering material utilized in the tobacco composition can vary, but is typically up to about 5 dry weight percent, and certain embodiments can be characterized by a pH adjuster/buffer content of at least about 0.5 dry weight percent, such as about 1 to about 5 dry weight percent.

Exemplary colorants include various dyes and pigments, such as caramel coloring and titanium dioxide. The amount of colorant utilized in the tobacco composition can vary, but is typically up to about 3 dry weight percent, and certain embodiments are characterized by a colorant content of at least about 0.1 dry weight percent, such as about 0.5 to about 3 dry weight percent.

Exemplary humectants include glycerin and propylene glycol. The amount of humectant utilized in the tobacco composition can vary, but is typically up to about 2 dry weight percent, and certain embodiments can be characterized by a humectant content of at least about 0.1 dry weight percent, such as about 0.2 to about 2 dry weight percent.

Other ingredients such as preservatives (e.g., potassium sorbate) or disintegration aids (e.g., microcrystalline cellulose, croscarmellose sodium, crospovidone, sodium starch glycolate, pregelatinized corn starch, and the like) can also be used. Typically, such ingredients are used in amounts of up to about 10 dry weight percent and usually at least about 0.1 dry weight percent, such as about 0.5 to about 10 dry weight percent.

Particularly with respect to smokeless tobacco compositions, tobacco compositions comprising lipophilic active agent infused tobacco leaves and/or tobacco materials of the disclosure can be formed into desired product shapes either before or after the infusion process. The method and apparatus used to form the tobacco composition will depend on the desired shape. Exemplary shapes include pill, tablet, sphere, sheet, coin, cube, bead, ovoid, obloid, bean, stick, and rod. For example, the tobacco composition can have the form of compressed tobacco pellets, multi-layered extruded pieces, extruded or formed rods or sticks, compositions having one type of tobacco formulation surrounded by a different type of tobacco formulation, rolls of tape-like films, readily water-dissolvable or water-dispersible films or strips (see, for example, U.S. Pat. Appl. Pub. No. 2006/0198873 to Chan et al.), or capsule-like materials possessing an outer shell (e.g., a pliable or hard outer shell that can be clear, colorless, translucent or highly colored in nature) and an inner region possessing tobacco or tobacco flavor (e.g., a Newtoniam fluid or a thixotropic fluid incorporating tobacco of some form).

Processed tobacco compositions comprising lipophilic active agent infused tobacco leaves and/or tobacco materials of the disclosure, such as compressed tobacco pellets, can be produced by compacting granulated tobacco and associated formulation components in the form of a pellet, and optionally coating each pellet with an overcoat material. Exemplary granulation devices are available as the FL-M Series granulator equipment (e.g., FL-M-3) from Vector Corporation and as WP 120V and WP 200VN from Alexanderwerk, Inc. Exemplary compaction devices, such as compaction presses, are available as Colton 2216 and Colton 2247 from Vector Corporation and as 1200i, 2200i, 3200, 2090, 3090 and 4090 from Fette Compacting. Devices for providing outer coating layers to compacted pelletized tobaccoformulations are available as CompuLab 24, CompuLab 36, Accela-Cota 48 and Accela-Cota 60 from Thomas Engineering.

Processed tobacco compositions comprising lipophilic active agent infused tobacco leaves and/or tobacco materials of the disclosure, such as multi-layered tobacco pellets, can be manufactured using a wide variety of extrusion techniques. For example, multi-layered tobacco pellets can be manufactured using co-extrusion techniques (e.g., using a twin screw extruder). In such a situation, successive wet or dry components or component mixtures can be placed within separate extrusion hoppers. Steam, gases (e.g., ammonia, air, carbon dioxide, and the like), and humectants (e.g., glycerin or propylene glycol) can be injected into the extruder barrel as each dry mix is propelled, plasticized, and cooked. As such, the various components are processed so as to be very well mixed, and hence, come in complete contact with each other. For example, the contact of components is such that individual components can be well embedded in the extrusion matrix or extrudate. See, for example, U.S. Pat. No. 4,821,749 to Toft et al., which is incorporated herein by reference. Multilayered materials can have the general form of films, and alternatively, multi-layered generally spherical materials can possess various layers extending from the inside outward.

Some shapes, such as rods or cubes, can be formed by first extruding the material through a die having the desired cross-section (e.g., round or square) and then optionally cutting the extruded material into desired lengths. Exemplary extrusion equipment suitable for use in the disclosure include industrial pasta extruders such as Model TP 200/300 available from Emiliomiti, LLC of Italy. Sheet-like materials can be prepared by applying the tobacco composition onto a moving belt and passing the moving belt through a nip formed by opposing rollers, followed by cutting the sheet into desired lengths.

### Bioavailability

Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation). Thus, many drugs may be metabolized before adequate plasma concentrations are reached.

Bioavailability is usually assessed by determining the area under the plasma concentration-time curve (AUC). AUC is directly proportional to the total amount of unchanged drug that reaches systemic circulation. Plasma drug concentration increases with extent of absorption; the maximum (peak) plasma concentration is reached when drug elimination rate equals absorption rate. Peak time is the most widely used general index of absorption rate; the slower the absorption, the later the peak time.

The bioavailability of some drugs is increased when co-administered with food, particularly agents such as cannabinoids that are Class II drugs under the Biopharmaceutical Drug Classification System (Kelepu et al. (2013) Acta Pharmaceutica Sinica B 3:361-372; Amidon et al. (1995) Pharm. Res. 12:413-420; Charman et al. (1997) J. Pharm. Sci. 86:269-282; Winstanley et al. (1989) Br. J. Clin. Pharmacol. 28:621-628). It is the lipid component of the food that plays a key role in the absorption of lipophilic drugs and that leads to enhanced oral bioavailability (Hunt & Knox (1968) J. Physiol. 194:327-336; Kelepu et al. (2013) Acta Pharmaceutica Sinica B 3:361-372). This has been attributed to the ability of a high fat meal to stimulate biliary and pancreatic secretions, to decrease metabolism and efflux activity, to increase intestinal wall permeability, and to a prolongation of gastrointestinal tract (GIT) residence time and transport via the lymphatic system (Wagnera et al. (2001) Adv. Drug Del. Rev. 50:S13-31; Kelepu et al. (2013) Acta Pharmaceutica Sinica B 3:361-372). High fat meals also elevate triglyceride-rich lipoproteins that associate with drug molecules and enhance intestinal lymphatic transport, which leads to changes in drug disposition and changes the kinetics of the pharmacological actions of poorly soluble drugs (Gershkovich et al. (2007) Eur. J. Pharm. Sci. 32:24-32; Kelepu et al. (2013) Acta Pharmaceutica Sinica B 3:361-372). However, co-administration of food with lipophilic drugs requires close control and/or monitoring of food intake when dosing such drugs, and can also be subject to problems with patient compliance (Kelepu et al. (2013) Acta Pharmaceutica Sinica B 3:361-372).

In other aspects, the bioavailability enhancing agent within the compositions and methods of the present disclosure is an edible oil or fat, a protective colloid, or both a protective colloid and an edible oil or fat. In another aspect, the bioavailability enhancing agent is also a lipophilic active agent taste masking agent. In another particular aspect, where the bioavailability enhancing agent is both a protective colloid, an edible oil or fat, and a lipophilic active agent taste masking agent, the bioavailability enhancing agent is nonfat dry milk. In a further aspect, the bioavailability enhancing agent is substantially free of omega-6 fatty acids. In other aspects, the bioavailability of the lipophilic active agent in a subject is at least about 1.5 times, 2 times, 5 times, or 10 times greater than the bioavailability of the lipophilic active agent in the subject in the absence of the bioavailability enhancing agent. In a further aspect, the bioavailability of the lipophilic active agent in a subject is greater than 20%.

An edible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C) mono-, di- or polyhydric alcohols. Some examples of digestible oils for use in this disclosure thus include: vegetable, nut, or seed oils (such as coconut oil, peanut oil, soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (such as fish liver oil, shark oil and mink oil).

In one aspect the edible oils disclosed herein serve as a primary bioavailability enhancing agents. The edible oils are obtained from one or more sources, for example, from plants, nuts or animals. In one aspect, the following are non-limiting examples of plant or vegetable oils, includes an edible oil selected from the group consisting of hemp seed oil, flaxseed oil, wheat germ oil, black currant seed oil, canola oil, castor oil, coconut oil, corn oil, cottonseed oil, grapeseed oil, olive oil, palm oil, palm kernel oil, rapeseed oil, safflower oil, soybean oil, sunflower oil, and any combination thereof,

In another aspect the edible oil is selected from the group consisting of canola oil, coconut oil, corn oil, cottonseed oil, olive oil, palm oil, palm kernel oil, rapeseed oil, safflower oil, soybean oil, sunflower oil, hydrogenated coconut oil, hydrogenated palm oil, hydrogenated soybean oil, and any combination thereof.

In a further aspect, the bioavailability enhancing agent is a long chain (C₁₄-C₂₂) fatty acid. In a further aspect, the bioavailability enhancing agent is a medium chain (C₇-C₁₃) fatty acid. In further aspects, the bioavailability enhancing agent is a combination of medium and long chain fatty acids.

Examples of protective colloids include polypeptides (such as gelatin, casein, and caseinate), polysaccharides (such as starch, dextrin, dextran, pectin, and gum arabic), as well as whole milk, skimmed milk, milk powder or mixtures of these. However, it is also possible to use polyvinyl alcohol, vinyl polymers, for example polyvinylpyrrolidone, (meth)acrylic acid polymers and copolymers, methylcellulose, carboxymethylcellulose, hydroxypropylcellulose and alginates. For further details, reference may be made to R. A. Morton, Fast Soluble Vitamins, Intern. Encyclopedia of Food and Nutrition, Vol. 9, Pergamon Press 1970, pages 128-131.

In one aspect the bioavailability agent is non-fat dry milk.

Oral administration constitutes the preferred route of administration for a majority of drugs. However, drugs that have an undesirable or bitter taste leads to lack of patient compliance in the case of orally administered dosage forms. In such cases, taste masking is an essential tool to improve patient compliance. Because lipophilic active agents (e.g., cannabinoids such as cannabidiol) have an undesirable taste profile, in order to improve compliance, the presently disclosed compositions also comprise one or more lipophilic active agent taste masking agents. Examples of lipophilic active agent taste-masking agents include dry milk as described above, as well as menthol, sweeteners, sodium bicarbonate, ion-exchange resins, cyclodextrin inclusion compounds, adsorbates, and the like.

In another aspect, taste-masking agents used with tobacco products include flavoring agents such as salts (e.g., sodium chloride, potassium chloride, sodium citrate, potassium citrate, sodium acetate, potassium acetate, and the like), natural sweeteners (e.g., fructose, sucrose, glucose, maltose, mannose, galactose, lactose, and the like), artificial sweeteners (e.g., sucralose, saccharin, aspartame, acesulfame K, neotame, and the like); and mixtures thereof. In other aspects, suitable flavoring agents include, but are not limited to, vanilla, vanillin, ethyl vanillin, orange oil, peppermint oil, strawberry, raspberry, and mixtures thereof.

In a further aspect, the bioavailability enhancing agent is substantially free of omega-6 fatty acids. As used herein, “substantially free” means largely but not wholly pure.

In other aspects, the bioavailability of the lipophilic active agent in a subject is at least about 1.5 times, 2 times, 2.5 times, 3 times, 3.5 times, 4 times, 4.5 times, 5 times, 5.5 times, 6 times, 6.5 times, 7 times, 7.5 times, 8 times, 8.5 times, 9 times, 9.5 times, or 10 times greater than the bioavailability of the lipophilic active agent in the subject in the absence of the bioavailability enhancing agent.

In a further aspect, the bioavailability of the lipophilic active agent in a subject is greater than 20% or at least about 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or greater.

Assays and methods for measuring lipophilic active agent bioavailability are well known in the art (see, e.g., Rocci & Jusko (1983) Comput. Programs Biomed. 16:203-215; Shargel & Yu (1999) Appled biopharmaceutics & pharnacokinetics (4th ed.). New York: McGraw-Hill, Hu & Li (2011) Oral Bioavailability: Basic Principles, Advanced Concepts, and Applications, John Wiley & Sons Ltd.; Karschner et al. (2011) Clinical Chemistry 57:66-75; Ohlsson et al. (1980) Clin. Pharmacol. Ther. 28:409-416; Ohlsson et al. (1982) Biomed. Environ. Mass Spectrom. 9:6-10; Ohlsson et al. (1986) Biomed. Environ. Mass Spectrom. 13:77-83; Karschner et al. (2010) Anal. Bioanal. Chem. 397:603-611).

### Lyophilization

In a further aspect, the lipophilic active agent infused tobacco leaves and/or tobacco materials of the present disclosure are lyophilized. Lyophilization, also known as freeze-drying, is a process whereby water is sublimed from a composition after it is frozen. The frozen solution is then typically subjected to a primary drying step in which the temperature is gradually raised under vacuum in a drying chamber to remove most of the water, and then to a secondary drying step typically at a higher temperature than employed in the primary drying step to remove the residual moisture in the lyophilized composition. The lyophilized composition is then appropriately sealed and stored for later use. Tang et al. (2004) Pharmaceutical Research 21:191-200 describes the scientific principles pertaining to freeze drying and guidelines for designing suitable freeze drying processes. Further description of freeze drying is found in Remington (2006) The Science and Practice of Pharmacy, 21^{st} edition, Lippincott Williams & Wilkins, pp. 828-831.

### Lipophilic Active Agents

### 1. Cannabinoids

Cannabis sativa L. is one of the most widely used plants for both recreational and medicinal purposes. Over 500 natural constituents have been isolated and identified from C. sativa covering several chemical classes (Ahmed et al. (2008) J. Nat. Prod 71:536-542; Ahmed et al. (2008) Tetrahedron Lett. 49:6050-6053; ElSohly & Slade (2005) Life Sci. 78:539-548; Radwan et al. (2009) J. Nat. Prod 72:906-911; Radwan et al. (2008) Planta Medica. 74:267-272; Radwan et al. (2008) J. Nat. Prod 69:2627-2633; Ross et al. (1995) Zagazig J. Pharm. Sci. 4:1-10; Turner et al. (1980) J. Nat. Prod 43:169-170). Cannabinoids belong to the chemical class of terpenophenolics, of which at least 85 have been uniquely identified in cannabis (Borgelt et al. (2013) Pharmacotherapy 33:195-209).

Cannabinoids are ligands to cannabinoid receptors (CB₁, CB₂) found in the human body (Pertwee (1997) Pharmacol. Ther. 74:129-180). The cannabinoids are usually divided into the following groups: classical cannabinoids; non-classical cannabinoids; aminoalkylindole-derivatives; and eicosanoids (Pertwee (1997) Pharmacol. Ther. 74:129-180). Classical cannabinoids are those that have been isolated from C. sativa L. or their synthetic analogs. Non-classical cannabinoids are bi- or tri-cyclic analogs of tetrahydrocannabinol (THC) (without the pyran ring). Aminoalkylindoles and eicosanoids are substantially different in structure compared to classical and non-classical cannabinoids. The most common natural plant cannabinoids (phytocannabinoids) are cannabidiol (CBD), cannabigerol (CBG), cannabichromene (CBC), and cannabinol (CBN). The most psychoactive cannabinoid is Δ⁹-THC.

In recent years, marijuana and its components have been reported in scientific literature to counter the symptoms of a broad range of conditions including but not limited to multiple sclerosis and other forms of muscular spasm; movement disorders; pain, including migraine headache; glaucoma; asthma; inflammation; insomnia; and high blood pressure. There may also be utility for cannabinoids as anxiolytics, anti-convulsives, anti-depressants, anti-psychotics, anti-cancer agents, as well as appetite stimulants. Pharmacological and toxicological studies of cannabinoids have largely been focused on a synthetic analog of Δ⁹-THC (commercially available under the generic name Dronabinol). In 1985, Dronabinol was approved by the FDA for the treatment of chemotherapy associated nausea and vomiting, and later for AIDS-associated wasting and anorexia.

Therapeutic use of cannabinoids has been hampered by the psychoactive properties of some compounds (e.g., Dronabinol) as well as their low bioavailability when administered orally. Bioavailability refers to the extent and rate at which the active moiety (drug or metabolite) enters systemic circulation, thereby accessing the site of action. The low bioavailability of orally ingested cannabinoids (from about 6% to 20%; Adams & Martin (1996) Addiction 91: 1585-614; Agurell et al. (1986) Pharmacol. Rev. 38: 21-43; Grotenhermen (2003) Clin. Pharmacokinet. 42: 327-60) has been attributed to their poor dissolution properties and extensive first pass metabolism.

Cannabinoids are a heteromorphic group of chemicals which directly or indirectly activate the body's cannabinoid receptors. There are three main types of cannabinoids: herbal cannabinoids that occur uniquely in the cannabis plant, synthetic cannabinoids that are manufactured, and endogenous cannabinoids that are produced in vivo. Herbal cannabinoids are nearly insoluble in water but soluble in lipids, alcohol, and non-polar organic solvents. These natural cannabinoids are concentrated in a viscous resin that is produced in glandular structures known as trichomes. In addition to cannabinoids, the resin is rich in terpenes, which are largely responsible for the odor of the cannabis plant.

The identification of Δ⁹-tetrahydrocannabinol (THC) as a major psychoactive drug and its chemical synthesis in 1964 opened a new era of synthetic cannabinoids as pharmacological agents. Cannabinoid research has increased tremendously in recent years since the discovery of cannabinoid receptors and the endogenous ligands for these receptors. The receptors include CB1, predominantly expressed in the brain, and CB2, primarily found on the cells of the immune system. Cannabinoid receptors belong to a superfamily of G-protein-coupled receptors. They are single polypeptides with seven transmembrane α-helices, and have an extracellular, glycosylated N-terminus and intracellular C-terminus. Both CB1 and CB2 cannabinoid receptors are linked to G1/0-proteins. In addition to these receptors, endogenous ligands for these receptors capable of mimicking the pharmacological actions of THC have also been discovered. Such ligands were designated endocannabinoids and included anandamide and 2-arachidonoyl glycerol (2-AG). Anandamide is produced in the brain and peripheral immune tissues such as the spleen.

Unlike THC, which exerts its action by binding to CB1 and CB2, cannabidiol does not bind to these receptors and hence has no psychotropic activity. Instead, cannabidiol indirectly stimulates endogenous cannabinoid signaling by suppressing the enzyme that breaks down anandamide (fatty acid amide hydroxylase, “FAAH”). Cannabidiol also stimulates the release of 2-AG. Cannabidiol has been reported to have immunomodulating and anti-inflammatory properties, to exhibit anticonvulsive, anti-anxiety, and antipsychotic activity, and to function as an efficient neuroprotective antioxidant.

Cannabinoids in cannabis are often inhaled via smoking, but may also be ingested. Smoked or inhaled cannabinoids have reported bioavailabilities ranging from 2-56%, with an average of about 30% (Huestis (2007) Chem. Biodivers. 4:1770-1804; McGilveray (2005) Pain Res. Manag. 10 Suppl. A:15A-22A). This variability is mainly due to differences in smoking dynamics. Cannabinoids that are absorbed through the mucous membranes in the mouth (buccomucosal application) have bioavailabilities of around 13% (Karschner et al. (2011) Clin. Chem. 57:66-75). By contrast, when cannabinoids are ingested, bioavailability is typically reduced to about 6% (Karschner et al. (2011) Clin. Chem. 57:66-75).

Accordingly, in other aspects, within the compositions and methods of the present disclosure, the lipophilic active agent is a cannabinoid.

In particular aspects, at least one cannabinoid within the compositions and methods of the present disclosure is selected from the group consisting of:

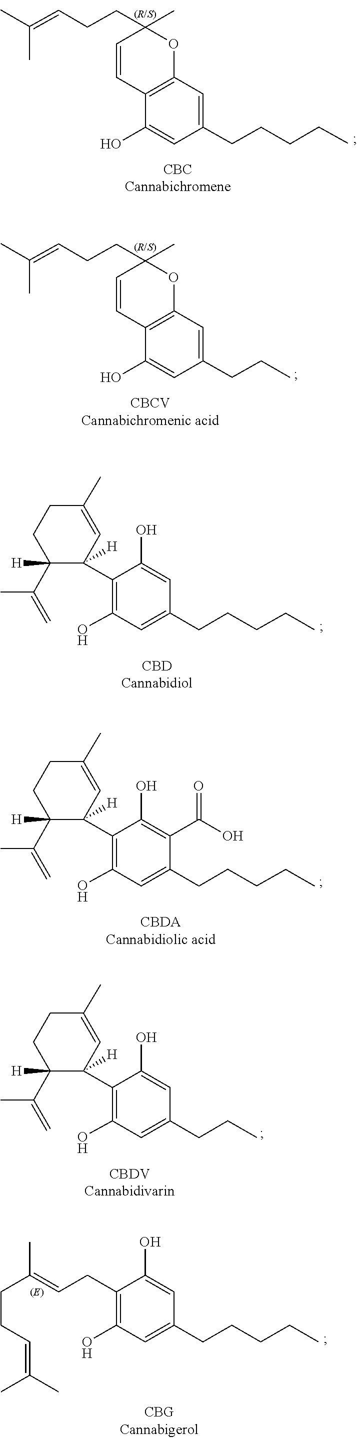

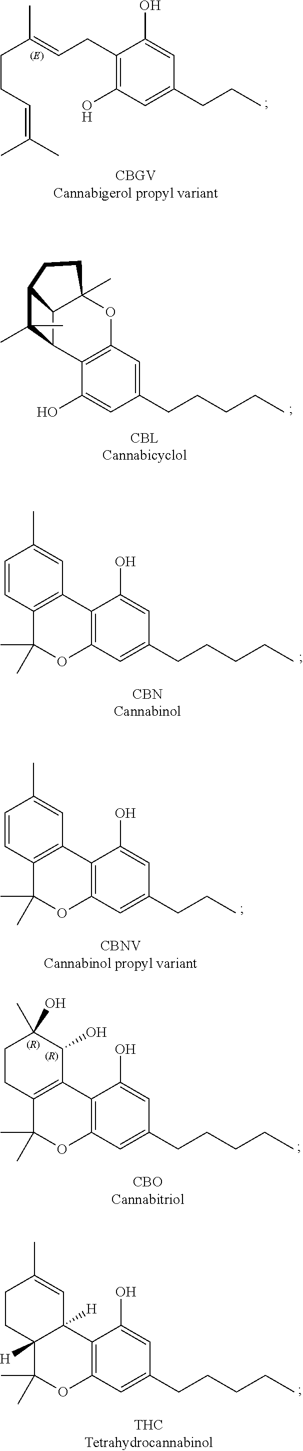

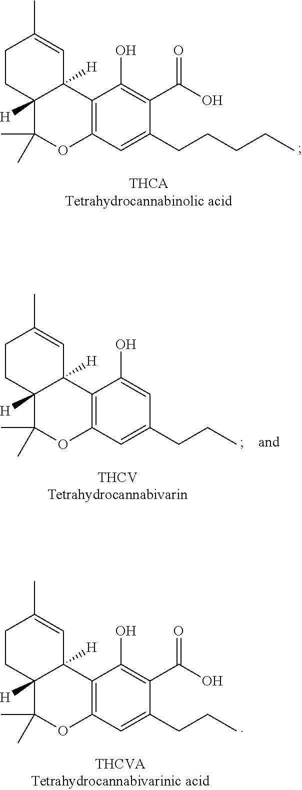

In particular aspects, at least one cannabinoid within the compositions and methods of the present disclosure is a non-psychoactive cannabinoid such as cannabidiol. In some particularly disclosed aspects, the cannabinoid is selected from the group consisting of:

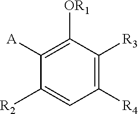

where A is aryl, and particularly

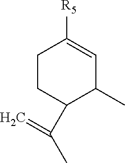

but not a pinene such as:

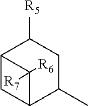

and the R₁-R₅ groups are each independently selected from the groups of hydrogen, lower substituted or unsubstituted alkyl, substituted or unsubstituted carboxyl, substituted or unsubstituted alkoxy, substituted or unsubstituted alcohol, and substituted or unsubstituted ethers, and R₆-R₇ are H or methyl. In particular aspects, there are no nitrogens in the rings, and/or no amino substitutions on the rings.

In other aspects, the cannabinoid is selected from the group consisting of:

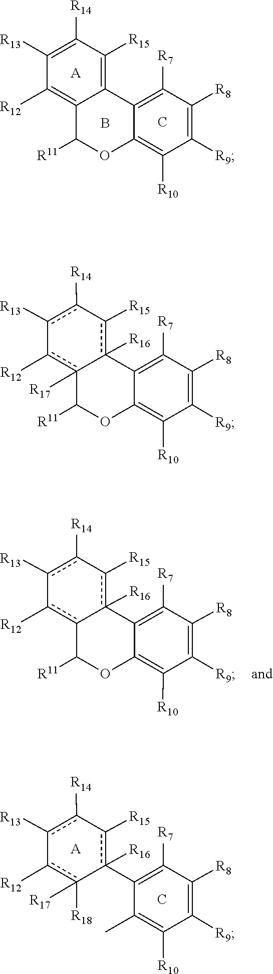

where there can be 0 to 3 double bonds on the A ring, as indicated by the optional double bonds indicated by dashed lines on the A ring. The C ring is aromatic, and the B ring can be a pyran. Particular aspects are dibenzo pyrans and cyclohexenyl benzenediols. Particular aspects of the cannabinoids of the present disclosure may also be highly lipid soluble, and in particular aspects can be dissolved in an aqueous solution only sparingly (for example 10 mg/ml or less). The octanol/water partition ratio at neutral pH in useful aspects is 5000 or greater, for example 6000 or greater. This high lipid solubility enhances penetration of the drug into the central nervous system (CNS), as reflected by its volume of distribution (V_{d}) of 1.5 L/kg or more, for example 3.5 L/kg, 7 L/kg, or ideally 10 L/kg or more, for example at least 20 L/kg. Particular aspects may also be highly water soluble derivatives that are able to penetrate the CNS, for example carboxyl derivatives.

R₇₋₁₈ are independently selected from the group of H, substituted or unsubstituted alkyl, especially lower alkyl, for example unsubstituted C₁-C₃ alkyl, hydroxyl, alkoxy, especially lower alkoxy such as methoxy or ethoxy, substituted or unsubstituted alcohol, and unsubstituted or substituted carboxyl, for example COOH or COCH₃. In other aspects R₇₋₁₈ can also be substituted or unsubstituted amino, and halogen.

In particular aspects, at least one cannabinoid within the compositions and methods of the present disclosure is a non-psychoactive cannabinoid, meaning that the cannabinoid has substantially no psychoactive activity mediated by the cannabinoid receptor (for example an IC₅₀ at the cannabinoid receptor of greater than or equal to 300 nM, for example greater than 1 μM and a Kᵢ greater than 250 nM, especially 500-1000 nM, for example greater than 1000 nM).

In other particular aspects, the cannabinoids within the compositions and methods of the present disclosure are selected from the group consisting of:

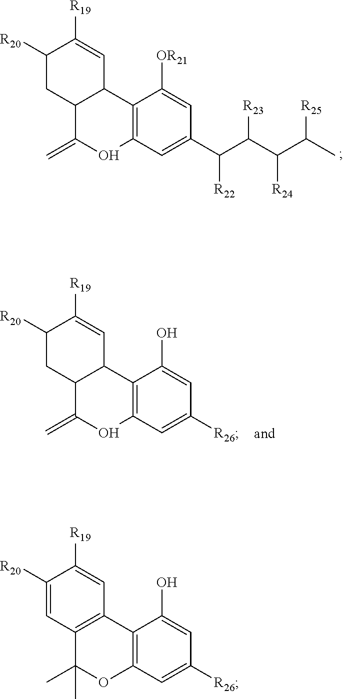

where R₁₉ is substituted or unsubstituted alkyl, such as lower alkyl (for example methyl), lower alcohol (such as methyl alcohol) or carboxyl (such as carboxylic acid) and oxygen (as in ═O); R₂₀ is hydrogen or hydroxy; R₂₁ is hydrogen, hydroxy, or methoxy; R₂₂ is hydrogen or hydroxy; R₂₃ is hydrogen or hydroxy; R₂₄ is hydrogen or hydroxy; R₂₅ is hydrogen or hydroxy; and R₂₆ is substituted or unsubstituted alkyl (for example n-methyl alkyl), substituted or unsubstituted alcohol, or substituted or unsubstituted carboxy.

In other particular aspects, the cannabinoids within the compositions and methods of the present disclosure are selected from the group consisting of:

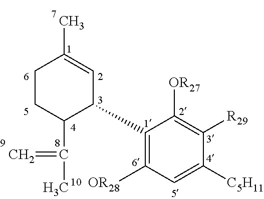

wherein numbering conventions for each of the ring positions are shown, and R₂₇, R₂₈ and R₂₉ are independently selected from the group consisting of H, unsubstituted lower alkyl such as CH₃, and carboxyl such as COCH₃. Particular examples of nonpsychoactive cannabinoids that fall within this definition are cannabidiol and

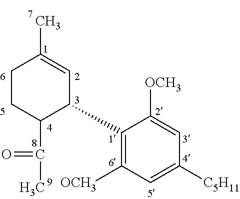

and other structural analogs of cannabidiol.

In other particular aspects, the cannabinoids within the compositions and methods of the present disclosure are selected from the group consisting of:

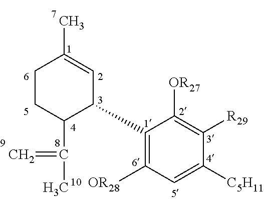

wherein R₂₇, R₂₈ and R₂₉ are independently selected from the group consisting of H, lower alkyl such as CH₃, and carboxyl such as COCH₃, and particularly wherein:
a) R₂₇═R₂₈═R₂₉═H
b) R₂₇═R₂₉═H; R₂₈═CH₃
c) R₂₇═R₂₈═CH₃; R₂₉═H
d) R₂₇═R₂₈═COCH₃; R₂₉═H
e) R₂₇═H; R₂₈═R₂₉═COCH₃

When R₂₇═R₂₈═R₂₉═H, then the compound is cannabidiol (CBD). When R₂₇═R₂₉═H and R₂₈═CH₃, the compound is CBD monomethyl ether. When R₂₇═R₂₈═CH₃ and R₂₉═H, the compound is CBD dimethyl ether. When R₂₇═R₂₈═COCH₃ and R₂₉═H, the compound is CBD diacetate. When R₂₇═H and R₂₈═R₂₉═COCH₃, the compound is CBD monoacetate.

### 2. Terpenes and Terpenoids

Terpenes are a diverse group of organic hydrocarbons derived from 5-carbon isoprene units and are produced by a wide variety of plants. Terpenoids are terpenes which have been chemically modified to add functional groups including heteroatoms. Terpenes and terpenoids are important building blocks for hormones, vitamins, pigments, steroids, resins, and essential oils. Terpenes are naturally present in cannabis; however, they can be removed during the extraction process. Terpenes and terpenoids have various pharmaceutical (pharmacodynamic) effects and can be selected for the desired pharmaceutical activities.

In one embodiment, the terpene/terpenoid includes limonene. Limonene is a colorless liquid hydrocarbon classified as a cyclic terpene. The more common D-isomer possesses a strong smell of oranges and a bitter taste. It is used in chemical synthesis as a precursor to carvone and as a solvent in cleaning products. Limonene is a chiral molecule. Biological sources produce one enantiomer—the principal industrial source—citrus fruit, contains D-limonene ((+)-limonene), which is the (R)-enantiomer (CAS number 5989-27-5, EINECS number 227-813-5). Racemic limonene is known as dipentene. Its IUPAC name is 1-methyl-4-(1-methylethenyl)-cyclohexene. It is also known as 4-isopropenyl-1-methylcyclohexenep-Menth-1,8-dieneRacemic: DL-limonene; dipentene.

Limonene has a history of use in medicine, food and perfume. It has very low toxicity, and humans are rarely allergic to it. Limonene is used as a treatment for gastric reflux and as an anti-fungal agent. Its ability to permeate proteins makes it a useful treatment for toenail fungus. Limonene is also used for treating depression and anxiety. Limonene is reported to assist in the absorption of other terpenoids and chemicals through the skin, mucous membranes and digestive tract. Limonene has immunostimulant properties. Limonene is also used as botanical insecticide

The principle metabolites of limonene are (+)- and (−)-trans-carveol, a product of 6-hydroxylation) and (+)- and (−)-perillyl alcohol, a product of 7-hydroxylation by CYP2C9 and CYP2C19 cytochromes in human liver microsomes. The enantiomers of perillyl alcohol have been researched for possible pharmacological possibilities as dietary chemotherapeutic agents. They are considered novel therapeutic options in some CNS neoplasms and other solid tumors, especially for treatment of gliomas. The cytotoxic activities of perillyl alcohol and limonene metabolites are likely due to their antiangiogenic properties, hyperthermia inducing effects, negative apoptosis regulation and effect on Ras pathways.

In another embodiment, the terpene/terpenoid includes linalool. Linalool is a naturally occurring terpene alcohol chemical found in many flowers and spice plants with many commercial applications, the majority of which are based on its pleasant scent (floral and slightly spicy). It is also known as P-linalool, linalyl alcohol, linaloyl oxide, p-linalool, allo-ocimenol, and 3,7-dimethyl-1,6-octadien-3-ol. Its IUPAC name is 3,7-dimethylocta-1,6-dien-3-ol.

More than 200 species of plants produce linalool, mainly in the families Lamiaceae, Lauraceae and Rutaceae. It has also been found in some fungi. Linalool has been used for thousands of years as a sleep aid. Linalool is an important precursor in the formation of Vitamin E. It has a history of use in the treatment of both psychosis and anxiety, and as an anti-epileptic agent. It also provides analgesic pain relief Its vapors have been shown to be an effective insecticide against fleas, fruit flies and cockroaches. Linalool is used as a scent in an estimated 60-80% of perfumed hygiene products and cleaning agents including soaps, detergents, shampoos and lotions.

In another embodiment, the terpene/terpenoid includes myrcene. Myrcene, or β-myrcene, is an olefinic natural organic compound. It is classified as a hydrocarbon, more precisely as a monoterpene. Terpenes are dimers of isoprene, and myrcene is one of the most important. Myrcene is a component of the essential oil of several plants including bay, cannabis, ylang-ylang, wild thyme, mango, parsley and hops. Myrcene is produced mainly semi-synthetically from myrcia, from which it gets its name. Myrcene is a key intermediate in the production of several fragrances. α-Myrcene is the name for the structural isomer 2-methyl-6-methylene-1,7-octadiene, which is not found in nature and is little used. Its IUPAC name is 7-methyl-3-methylene-1,6-octadiene.

Myrcene has an analgesic effect and is likely to be responsible for the medicinal properties of lemon grass tea. It has anti-inflammatory properties through Prostaglandin E2. The analgesic action can be blocked by naloxone or yohimbine in mice, which suggests mediation by alpha 2-adrenoceptor stimulated release of endogenous opioids. β-Myrcene is reported to have anti-inflammatory properties, and is used to treat spasms, sleep disorders and pain. Myrcene appears to lower resistance across the blood to brain barrier, allowing itself and many other chemicals to cross the barrier more effectively.

In another embodiment, the terpene/terpenoid includes α-Pinene. α-Pinene is one of the primary monoterpenes that is physiologically critical in both plants and animals. It is an alkene and it contains a reactive four-membered ring. α-Pinene tends to react with other chemicals, forming a variety of other terpenes including D-limonene and other compounds. α-Pinene has been used for centuries as a bronchodilator in the treatment of asthma. It is highly bioavailable with 60% human pulmonary uptake with rapid metabolism. α-Pinene is an anti-inflammatory via PGE1, and appears to be a broad-spectrum antibiotic. It acts as an acetylcholinesterase inhibitor, aiding memory. Products of α-pinene which have been identified include pinonaldehyde, norpinonaldehyde, pinic acid, pinonic acid, and pinalic acid.

Pinene is found in conifer, pine and orange. α-Pinene is a major constituent in turpentine. Its IUPAC name is (1S,5S)-2,6,6-Trimethylbicyclo[3.1.1]hept-2-ene ((−)-α-Pinene).

In another embodiment, the terpene/terpenoid includes β-Pinene. β-Pinene is one of the most abundant compounds released by trees. It is one of the two isomers of pinene, the other being α-pinene. It is a common monoterpene, and if oxidized in air, the allylic products of the pinocarveol and myrtenol family prevail. Its IUPAC name is 6,6-dimethyl-2-methylenebicyclo[3.1.1]heptane and is also known as 2(10)-Pinene; Nopinene; Pseudopinene. It is found in cumin, lemon, pine and other plants.

In another embodiment, the terpene/terpenoid includes caryophyllene, also known as β-caryophyllene. Caryophyllene is a natural bicyclic sesquiterpene that is a constituent of many essential oils, including clove, cannabis, rosemary and hops. It is usually found as a mixture with isocaryophyllene (the cis double bond isomer) and α-humulene, a ring-opened isomer. Caryophyllene is notable for having a rare cyclobutane ring. Its IUPAC name is 4,11,11-trimethyl-8-methylene-bicyclo[7.2.0]undec-4-ene.

Caryophyllene is known to be one of the compounds that contribute to the spiciness of black pepper. In a study conducted by the Swiss Federal Institute of Technology, β-caryophyllene was shown to be selective agonist of cannabinoid receptor type-2 (CB2) and to exert significant cannabimimetic, anti-inflammatory effects in mice. Anti-nociceptive, neuroprotective, anxiolytic, antidepressant and anti-alcoholic activity have been tied to caryophyllene. Because β-caryophyllene is an FDA approved food additive, it is considered the first dietary cannabinoid.

In another embodiment, the terpene/terpenoid includes citral. Citral, or 3,7-dimethyl-2,6-octadienal or lemonal, is either a pair, or a mixture of terpenoids with the molecular formula C₁₀H₁₆O. The two compounds are double bond isomers. The E-isomer is known as geranial or citral A. The Z-isomer is known as neral or citral B. Its IUPAC name is 3,7-dimethylocta-2,6-dienal. It is also known as citral, geranial, neral, geranialdehyde.

Citral is present in the oils of several plants, including lemon myrtle, lemongrass, verbena, lime, lemon and orange. Geranial has a pronounced lemon odor. Neral's lemon odor is not as intense, but sweet. Citral is primarily used in perfumery for its citrus quality. Citral is also used as a flavor and for fortifying lemon oil. It has strong antimicrobial qualities, and pheromonal effects in insects. Citral is used in the synthesis of vitamin A, ionone and methylionone.

In another embodiment, the terpene/terpenoid includes humulene. Humulene, also known as α-humulene or α-caryophyllene, is a naturally occurring monocyclic sesquiterpene (C₁₅H₂₄), which is an 11-membered ring consisting of 3 isoprene units containing three nonconjugated C═C double bonds, two of them being triply substituted and one being doubly substituted. It was first found in the essential oils of Humulus lupulus (hops). Humulene is an isomer of β-caryophyllene, and the two are often found together as a mixture in many aromatic plants.

Humulene has been shown to produce anti-inflammatory effects in mammals, which demonstrates potential for management of inflammatory diseases. It produces similar effects to dexamethasone, and was found to decrease the edema formation caused by histamine injections. Humulene produced inhibitory effects on tumor necrosis factor-α (TNFα) and interleukin-1.beta. (IL1B) generation in carrageenan-injected rats. In Chinese medicine, it is blended with β-caryophyllene and used as a remedy for inflammation.

Other exemplary terpenes and terpenoids include menthol, eucalyptol, borneol, pulegone, sabinene, terpineol, and thymol. In one embodiment, an exemplary terpene/terpenoid is eucalyptol.

### 3. Non-Steroidal Anti-Inflammatory Drugs (NSAIDs)

NSAIDs are the second-largest category of pain management treatment options in the world. The global pain management market was estimated at $22 billion in 2011, with $5.4 billion of this market being served by NSAID's. The U.S. makes up over one-half of the global market. The opioids market (such as morphine) form the largest single pain management sector but are known to be associated with serious dependence and tolerance issues.

Although NSAIDs are generally a safe and effective treatment method for pain, they have been associated with a number of gastrointestinal problems including dyspepsia and gastric bleeding.

Delivery of NSAIDs through the compositions and methods of the present disclosure will provide the beneficial properties of pain relief with lessened negative gastrointestinal effects, and also deliver lower dosages of active ingredients in order to provide pain management outcomes across a variety of indications.

Accordingly, in other aspects, within the compositions and methods of the present disclosure, the lipophilic active agent is an NSAID, particularly wherein the NSAID is selected from the group consisting of acetylsalicylic acid, ibuprophen, acetaminophen, diclofenac, indomethacin, and piroxicam.

### 4. Vitamins

The global vitamin and supplement market is worth $68 billion according to Euromonitor. The category is both broad and deep, comprised of many popular and some lesser known substances. Vitamins in general are thought to be an $8.5 billion annual market in the U.S. The U.S. is the largest single national market in the world, and China and Japan are the 2^{nd} and 3^{rd} largest vitamin markets.

Vitamin E is fat soluble and can be incorporated into cell membranes which can protect them from oxidative damage. Global consumption of natural source vitamin E was 10,900 metric tons in 2013 worth $611.9 million.

Delivery of fat soluble vitamins through the compositions and methods of the present disclosure will result in less waste and lower dosages of administration. In addition, ingestion of pills is an unpleasant experience for many people so vitamin delivery through common food groups will vastly expand demand and use.

Accordingly, in other aspects, within the compositions and methods of the present disclosure, the lipophilic active agent is a vitamin, particularly wherein the vitamin is vitamin E.

### 5. Nicotine Compounds

Nicotine is a natural ingredient in tobacco leaves where it acts as a botanical insecticide (Hukkanen et al. (2005) Pharmacological Reviews 57:79-115). Comprising about 95% of the total alkaloid content of commercial cigarette tobacco, nicotine comprises about 1.5% by weight of commercial cigarette tobacco (Hukkanen et al. (2005) Pharmacological Reviews 57:79-115). Although oral snuff and pipe tobacco contain concentrations of nicotine similar to cigarette tobacco, cigar and chewing tobacco typically contain only about half of the nicotine concentration of cigarette tobacco (Hukkanen et al. (2005) Pharmacological Reviews 57:79-115). An average tobacco rod typically contains 10 to 14 mg of nicotine (Hukkanen et al. (2005) Pharmacological Reviews 57:79-115), and on average about 1 to 1.5 mg of nicotine is absorbed systemically during smoking (Hukkanen et al. (2005) Pharmacological Reviews 57:79-115). The nicotine in tobacco is largely the levorotary (S)-isomer, only 0.1 to 0.6% of total nicotine content is (R)-nicotine (Hukkanen et al. (2005) Pharmacological Reviews 57:79-115). The (R)-nicotine content of tobacco smoke is higher, with up to 10% of nicotine in smoke reported to be (R)-isomer, and thought to be attributed to racemization occurring during combustion (Hukkanen et al. (2005) Pharmacological Reviews 57:79-115).

More than 99% of all nicotine that is consumed worldwide is delivered through smoking cigarettes. Approximately 6,000,000 deaths per year, worldwide, are attributed primarily to the delivery of nicotine through the act of smoking according to the Centers for Disease Control and Prevention, which also estimates that over $170 billion per year is spent just in the U.S. on direct medical care costs for adult smokers. In any twelve month period, 69% of U.S. adult smokers want to quit smoking and 43% of U.S. adult smokers have attempted to quit.

Worldwide, retail cigarette sales were worth $722 billion in 2013, with over 5.7 trillion cigarettes sold to more than 1 billion smokers. It would be desirable in the art to provide further methods for altering the character and nature of tobacco (and tobacco compositions and formulations) useful in smoking articles and/or or smokeless tobacco products, including enhancement of bioavailability of active agents, masking of unpleasant tastes, and the incorporation of additional active agents. Furthermore, the delivery of nicotine to satisfy current demand via the compositions and methods of the present disclosure, can in part alleviate the consumer demand for cigarettes. Since most of the adverse health outcomes of nicotine consumption are associated with the delivery method and only to a lesser degree to the actual ingestion of nicotine, a vast positive community health outcome can be achieved through the reduction in smoking cigarettes.

Accordingly, in other aspects, within the compositions and methods of the present disclosure, the lipophilic active agent is a nicotine compound. Tobacco alkaloids include nicotine and nicotine-like or related pharmacologically active compounds such as nor-nicotine, lobeline and the like, as well as the free base substance nicotine and all pharmacologically acceptable salts of nicotine, including acid addition salts. “Nicotine compounds” as that term is used herein therefore includes all the foregoing tobacco alkaloids, as well as nicotine salts including but not limited to nicotine hydrogen tartrate and nicotine bitartrate dihydrate, as well as nicotine hydrochloride, nicotine dihydrochloride, nicotine sulfate, nicotine citrate, nicotine zinc chloride monohydrate, nicotine salicylate, nicotine oil, nicotine complexed with cyclodextrin, polymer resins such as nicotine polacrilex, nicotine resinate, and other nicotine-ion exchange resins, either alone or in combination. The nicotine compounds also include nicotine analogs that include, but are not limited to the structures shown below for (s)-Nicotine, Nornicotine, (S)-Cotinine, B-Nicotyrine, (S)-Nicotene-N′-Oxide, Anabasine, Anatabine, Myosmine, B-Nornicotyrine, 4-(Methylamino)-1-(3-pyridyl)-1-butene (Metanicotine) cis or trans, N′-Methylanabasine, N′Methylanatabine, N′Methylmyosmine, 4-(Methylamino)-1-(3-pyridyl)-1-butanone (Pseudoxynicotine), and 2,3′-Bipyridyl (Hukkanen et al. (2005) Pharmacological Reviews 57:79-115):

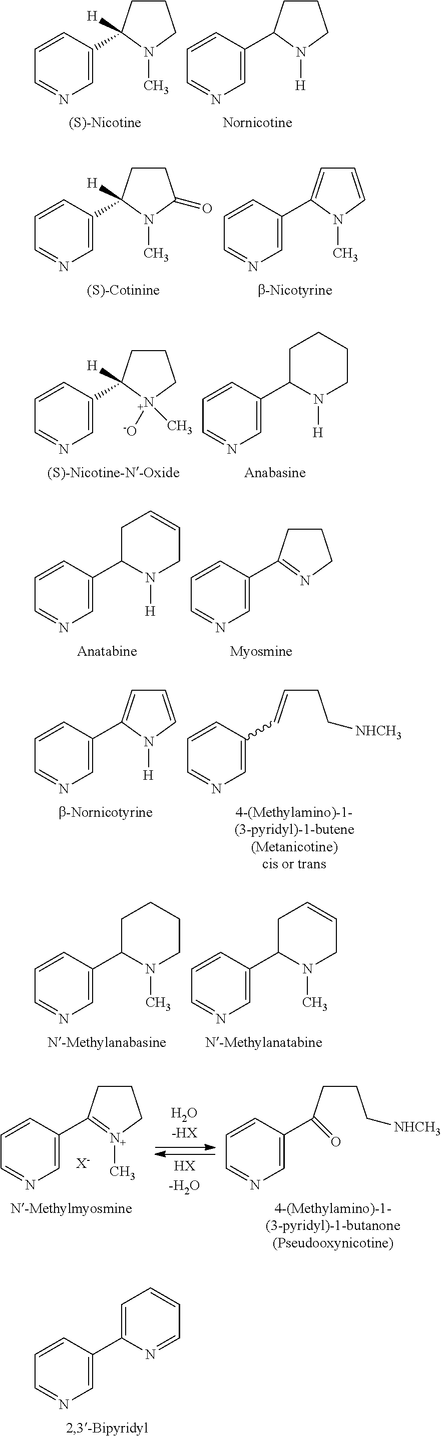

The nicotine compound may be used in one or more distinct physical forms well known in the art, including free base forms, encapsulated forms, ionized forms and spray-dried forms.

For additional description regarding the chemistry, absorption, metabolism, kinetics and biomarkers of nicotine is described in Hukkanen et al. (2005) Pharmacological Reviews 57:79-115 and Benowitz et al. (2009) Handb. Exp. Pharmacol. 192:29-60, which are both attached as Appendix C and incorporated herein in their entireties.

### 6. Phosphodiesterase Type 5 Inhibitors

Phosphodiesterase type 5 inhibitors (PDE5 inhibitors) block the degradative action of cGMP-specific phosphodiesterase type 5 (PDE5) on cyclic GMP in the smooth muscle cells lining the blood vessels supplying the corpus cavernosum of the penis. These drugs, including vardenafil (Levitra®), sildenafil (Viagra®), and tadalafil (Cialis®), are administered orally for the treatment of erectile dysfunction and were the first effective oral treatment available for the condition.

PDE5 inhibitors have also been studied for other clinical use as well, including cardiovascular and heart diseases. For example, because PDE5 is also present in the arterial wall smooth muscle within the lungs, PDE5 inhibitors have also been explored for lung diseases such as pulmonary hypertension and cystic fibrosis. Pulmonary arterial hypertension, a disease characterized by sustained elevations of pulmonary artery pressure, which leads to an increased incidence of failure of the right ventricle of the heart, which in turn can result in the blood vessels in the lungs become overloaded with fluid. Two oral PDE5 inhibitors, sildenafil (Revatio®) and tadalafil (Adcirca®), are approved for the treatment of pulmonary arterial hypertension. PDE5 inhibitors have been found to have activity as both a corrector and potentiator of CFTR protein abnormalities in animal models of cystic fibrosis disease (Lubamba et al., Am. J. Respir. Crit. Care Med. (2008) 177:506-515, Lubamba et al., J. Cystic Fibrosis (2012) 11:266-273). Sildenafil has also been studied as a potential anti-inflammatory treatment for cystic fibrosis. Oral PDE5 inhibitors have also been reported to have anti-remodeling properties and to improve cardiac inotropism, independent of afterload changes, with a good safety profile (Giannetta et al., BMC Medicine (2014) 12:185). However, oral administration of PDE5 inhibitors results in poor and variable bioavailability and also extensive metabolism in the liver (Sandqvist et al., Eur. J. Clin. Pharmacol. (2013) 69:197-207; Mehrotra, Intl. J. Impotence Res. (2007) 19:253-264). If oral doses are increased beyond certain levels, the incidence of systemic side effects increase which prevents the acceptable use of these drugs. (Levitra EMEA Scientific Discussion Document, 2005).

Accordingly, in other aspects, within the compositions and methods of the present disclosure, the PDE5 inhibitor may include, but is not limited to, avanafil, lodenafil, mirodenafil, sildenafil (or analogs thereof, for example, actetildenafil, hydroxyacetildenafil, or dimethyl-sildenafil), tadalafil, vardenafil, udenafil, acetildenafil, or thiome-thisosildenafil. The structures of these compounds are respectively shown below:

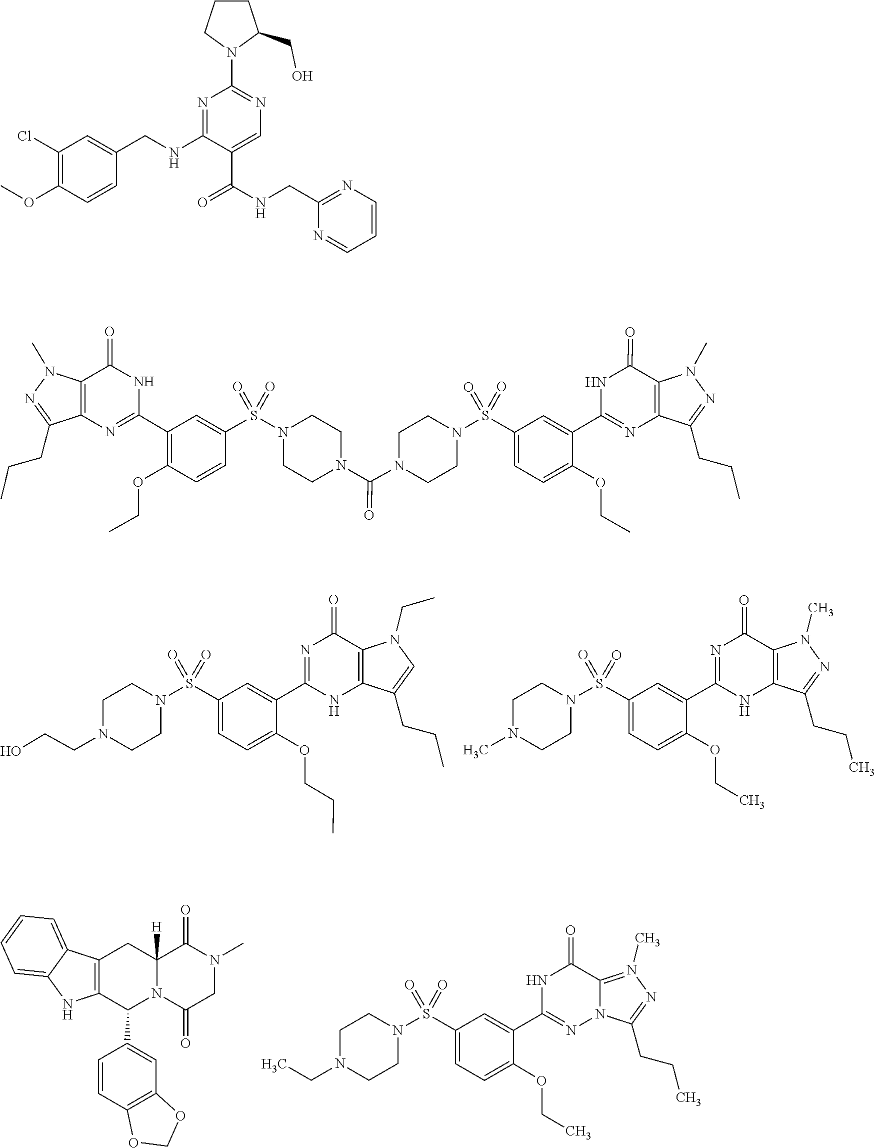

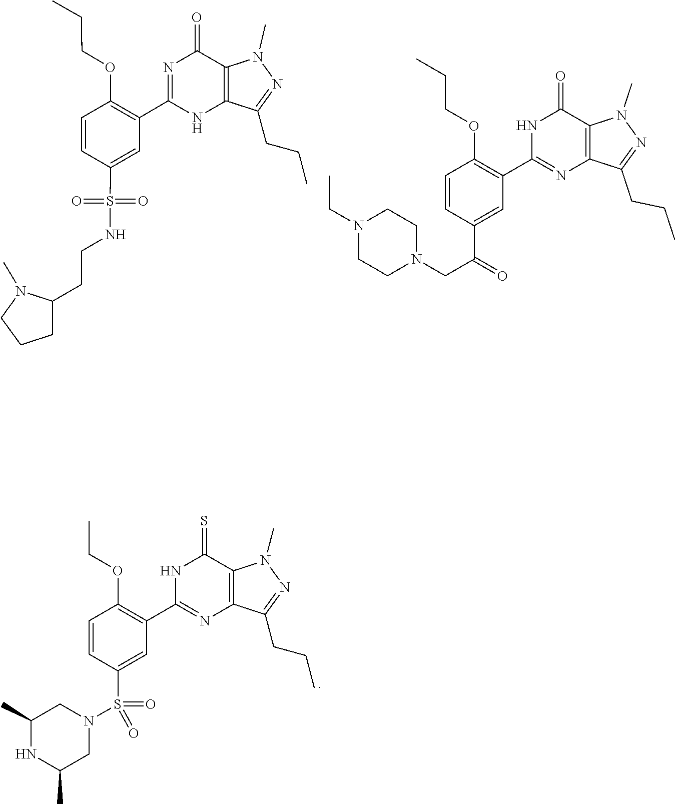

### 7. Maca Extract

Lepidium meyenii (Maca, maca-maca, maino, ayak chichira, and ayak willku) is a Peruvian plant of the Brassicaceae family cultivated for more than 2000 years. Its main active principles are alkaloids (Macaridine, Lepidiline A and B); bencil-isotiocyanate and glucosinolates; macamides, beta-ecdysone and fitosterols. These substances activate ATP synthesis which confers energizing properties. They also diminish variations in homeostasis produced by stress because they reduce corticosterone's high levels; prevent glucose diminution and the increase of suprarenal glands' weight due to stress. They also restore homeostasis and improve energy (Lopez-Fando et al. (2004) Phytother Res. 18:471-4). A double blind placebo-controlled, randomized, parallel trial study in which active treatment with different doses of Lepidium meyenii was compared with placebo showed an improvement in sexual desire. (Gonzales et al. (2002) Andrologia 34:367-72). Lepidium meyenii also improves sperm production and sperm motility by mechanisms not related to LH, FSH, PRL, T and E2 (Gonzales et al. (2001) Asian J. Androl. 3:301-3).

### 8. Estrogen

As used herein, “estrogen” includes estrogenic steroids such as estradiol (17-β-estradiol), estradiol benzoate, estradiol 17β-cypionate, estropipate, equilenin, equilin, estriol, estrone, ethinyl estradiol, conjugated estrogens, esterified estrogens, and mixtures thereof.

Estrogens refer to a group of endogenous and synthetic hormones that are important for and used for tissue and bone maintenance. Estrogens are endocrine regulators in the cellular processes involved in the development and maintenance of the reproductive system. The role of estrogens in reproductive biology, the prevention of postmenopausal hot flashes, and the prevention of postmenopausal osteoporosis are well established. Estradiol is the principal endogenous human estrogen, and is found in both women and men.

The biological actions of estrogens and antiestrogens are manifest through two distinct intracellular receptors, estrogen receptor alpha (ERα) and estrogen receptor beta (ERβ). Endogenous estrogens are typically potent activators of both receptor subtypes. For example estradiol acts as an ERα agonist in many tissues, including breast, bone, cardiovascular and central nervous system tissues. Selective estrogen receptor modulators commonly act differently in different tissues. For example, a SERM may be an ERα antagonist in the breast, but may be a partial ERα agonist in the uterus, bone and cardiovascular systems. Compounds that act as estrogen receptor ligands are, therefore, useful in treating a variety of conditions and disorders.

### 9. Progesterone and Progestins

The term “progesterone” as used herein refers to a member of the progestin family and comprises a 21 carbon steroid hormone. Progesterone is also known as D4-pregnene-3,20-dione; 4-pregnene-3,20-dione; or pregn-4-ene-3,20-dione. A progestin is a molecule whose structure is related to that of progesterone, is synthetically derived, and retains the biologically activity of progesterone. Representative synthetic progestin include, but are not limited to, modifications that produce 17a-OH esters (i.e., 17 a-hydroxyprogesterone caproate), as well as, modifications that introduce 6 a-methyl, 6-Me, 6-ene, and 6-chloro substituents onto progesterone (i.e., medroxyprogesterone acetate, megestrol acetate, and chlomadinone acetate).

### 10. Testosterone

Testosterone is the main androgenic hormone formed in the testes. Testosterone therapy is currently indicated for the treatment of male hypogonadism. It is also under investigation for the treatment of wasting conditions associated with AIDS and cancer, testosterone replacement in men over the age of 60, osteoporosis, combination hormone replacement therapy for women and male fertility control.

Orally administered testosterone is largely degraded in the liver, and is therefore not a viable option for hormone replacement since it does not allow testosterone to reach systemic circulation. Further, analogues of testosterone modified to reduce degradation (e.g., methyltestosterone and methandrostenolone) have been associated with abnormalities in liver function, such as elevation of liver enzymes and conjugated bilirubin. Injected testosterone produces wide peak-to-trough variations in testosterone concentrations that do not mimic the normal fluctuations of testosterone, and makes maintenance of physiological levels in the plasma difficult. Testosterone injections are also associated with mood swings and increased serum lipid levels. Injections require large needles for intramuscular delivery, which leads to diminished patient compliance due to discomfort.

To overcome these problems, transdermal delivery approaches have been developed to achieve therapeutic effects in a more patient friendly manner. For example, U.S. Pat. No. 5,460,820 discloses a testosterone-delivering patch for delivering 50 to 500 μg/day of testosterone to a woman. In addition, U.S. Pat. No. 5,152,997 discloses a device comprising a reservoir of testosterone with a skin permeation enhancer and a means for maintaining the reservoir in diffusional communication with the skin, such as an adhesive carrier device or a basal adhesive layer.

### 11. Fentanyl

Fentanyl (also known as fentanil) is a potent synthetic narcotic analgesic with a rapid onset and short duration of action. Fentanyl is a strong agonist at μ-opioid receptors. Fentanyl is manufactured under the trade names of SUBLIMAZE, ACTIQ, DUROGESIC, DURAGESIC, FENTORA, ONSOLIS INSTANYL, ABSTRAL, and others. Historically, fentanyl has been used to treat chronic breakthrough pain and is commonly used before procedures as an anesthetic in combination with a benzodiazepine. Fentanyl is approximately 100 times more potent than morphine with 100 micrograms of fentanyl approximately equivalent to 10 mg of morphine and 75 mg of pethidine (meperidine) in analgesic activity.

Suitable analogues of fentanyl include, without limitation, the following: alfentanil (trade name ALFENTA), an ultra-short-acting (five to ten minutes) analgesic; sufentanil (trade name SUFENTA), a potent analgesic for use in specific surgeries and surgery in heavily opioid-tolerant/opioid-dependent patients; remifentanil (trade name ULTIVA), currently the shortest-acting opioid, has the benefit of rapid offset, even after prolonged infusions; carfentanil (trade name WILDNIL) an analogue of fentanyl with an analgesic potency 10,000 times that of morphine and is used in veterinary practice to immobilize certain large animals such as elephants; and lofentanil an analogue of fentanyl with a potency slightly greater than carfentanil.

### 12. Buprenorphine

Buprenorphine (17-(cyclopropyl-methyl)-α-(1,1-dimethylethyl)-4,5-epoxy-18,19-dihy-dro-3-hydroxy-6-methoxy-α-methyl-6,14-ethenomorphinan-7-methanol) is an endoethylene morphinan derivative and a partial agonist of μ-opioid receptors with a strong analgesic effect. Buprenorphine is a partially synthetic opiate whose advantage over other compounds from this class of substance lies in a higher activity. This means that freedom from pain can be achieved in cancer or tumour patients with very unfavourable diagnosis, in the final stage, with daily doses of around 1 mg. A feature of buprenorphine in this context over the synthetic opioid fentanyl and its analogues is that the addictive potential of buprenorphine is lower than that of these compounds. A disadvantage is that, owing to the high molecular weight of buprenorphine, namely 467.64 daltons, it has been traditionally been difficult to effect its transdermal absorption.

### 13. Scopolamine

Scopolamine is a so-called antiemitic, it is preferably used to avoid nausea and vomiting, for example, arising from repeated passive changes in the balance occurring during traveling. Scopolamine is represented by the following chemical structure:

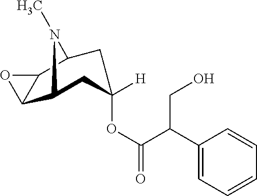

Scopolamine analogs are also encompassed by the compositions and methods of the present disclosure. It is understood that the phrase “scopolamine analogs” includes compounds that generally have the same backbone as scopolamine, but where various moieties have been substituted or replaced by other substituents or moieties. Some examples of scopolamine analogs that can be used in the compositions and methods disclosed herein include, but are not limited to, salts of scopolamine with various acids, such as hydrochloric acid, hydrobromic acid, hydroiodic acid, nitric acid, phosphoric acid, sulfuric acid, and the like. In one aspect, a suitable scopolamine analog can be scopolamine hydrobromide.

Additional examples of scopolamine analogs include, but are not limited to, N-alkylated analogs of scopolamine, that is, analogs containing an alkyl substituent attached to the nitrogen atom, forming a quaternary ammonium species. By “alkyl” is meant a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and the like. The alkyl group can also be substituted or unsubstituted.

Also included are other salts (e.g., pharmaceutically acceptable salts) of such N-alkylated scopolamine analogs.

Still further examples of scopolamine analogs include, but are not limited to, un-epoxylated analogs of scopolamine, that is, analogs where the epoxy group is removed. One example of such an analog is atropine. Like scopolamine, atropine has various salt and N-alkylated analogs. These atropine analogs are intended to be included by the phrase “scopolamine analogs.” As such, further examples of scopolamine analogs include, but are not limited to, analogs of atropine with various salts (e.g., atropine hydrobromide, atropine hydrochloride, and the like) and N-alkylated analogs of atropine (e.g., atropine methyl bromide). Also included are homatropine and its salts and N-alkylated analogs.

A list of suitable scopolamine analogs that can be used in the disclosed compositions and methods, including their commercial brand names, includes, but is not limited to, atropine, atropine hydrobromide, atropine oxide hydrochloride, atropine sulfate, belladonna, scopolamine, scopolamine hydrobromide, scopolamine methylbromide, scopolamine butylbromide, homatropine, ipratropium, tiotropium, hyoscyamine sulfate, methscopolamine, methscopolamine bromide, homatropine hydrobromide, homatropine methylbromide, hyoscyamine, hyoscyamine hydrobromide, hyoscyamine sulfate, propantheline bromide, anisotropine, anisotropine methylbromide, methantheline bromide, emepronium bromide, clindinium, clidinium bromide, hyoscine, hyoscine butylbromide, hyoscine hydrobromide, hyoscine methobromide, hyoscine methonitrite, hyoscyamine, hyoscyamine sulfate, buscapine, buscolysin, buscopan, butyiscopolamine, hyoscine N-butylbromide, N-butylscopolammonium bromide, scopolan bromide, butylscopolammonium bromide, N-butylscopolammonium chloride, hyoscine N-butylbromide, DD-234, hyoscine methiodide, hyoscine methobromide, methyiscopolamine nitrate, methylscopolammoium methylsulfate, N-methylscine methylsulfate, N-methylscopolamine bromide, N-methylscopolamine iodide, N-methylscopolamine methylchloride, N-methylscopolamine methylsulfate, N-methylscopolamine nitrate, skopyl, ulix bromide, N-methylscopolamine, N-methylscopolamine methobromide, scopolamine methylchloride, N-methylscine methylsulfate, tematropium methylsulfate, and N-isopropylatropine, including salts and derivatives thereof.

### Process

In other aspects, a process for making lipophilic active agent infused tobacco leaves and/or tobacco materials is provided comprising the steps of:
(a) contacting tobacco leaves and/or tobacco materials with an oil comprising a lipophilic active agent and a bioavailability enhancing agent, wherein the bioavailability enhancing agent comprises an edible oil comprising long chain fatty acids; and
(b) dehydrating the tobacco leaves and/or tobacco materials;
thereby producing lipophilic active agent infused tobacco leaves and/or tobacco materials;
wherein the lipophilic active agent infused tobacco leaves and/or tobacco materials comprise a therapeutically effective amount of the lipophilic active agent, and further wherein:
(i) the lipophilic active agent is selected from the group consisting of cannabinoids, terpenes and terpenoids, NSAIDs, vitamins, nicotine compounds, phosphodiesterase type 5 (PDE5) inhibitors, Maca extract, estrogen, progestin, testosterone, buprenorphine, and scopolamine; and
(ii) the bioavailability enhancing agent enhances the bioavailability of the lipophilic active agent.

In another aspect, step (a) further comprises saturating the food product in the oil comprising the lipophilic active agent and the bioavailability enhancing agent. In another aspect, step (a) further comprises contacting the tobacco leaves and/or tobacco materials with a flavoring agent, particularly wherein the flavoring agent is selected from the group consisting of vanilla, vanillin, ethyl vanillin, orange oil, peppermint oil, strawberry, raspberry, and mixtures thereof. In another aspect, the process further comprises a step of lyophilizing the lipophilic active agent infused tobacco leaves and/or tobacco materials.

In further aspects, the disclosed processes and methods use dehydration methods using dielectric energy, particularly microwave energy. In some aspects, the dielectric energy is selected from the group consisting of radio frequency energy, low frequency microwave energy, and high frequency microwave energy. In some aspects, the dehydration methods further comprise using dielectric energy under vacuum. In still further aspects, the dehydration methods further comprise stirring at a temperature of less than 70° C. In still further aspects, the disclosed processes and methods use dehydration methods using spray drying technology (e.g., methods of producing dry powders from a liquid or slurry by rapidly drying with a hot gas; see generally Mujumdar (2007) Handbook of Industrial Drying, CRC Press).

### Methods of Treatment

In a further aspect, a method of treating a condition is provided, comprising administering any of the compositions disclosed herein to a subject in need thereof.

Disclosed herein are methods for treating a subject having a disease or condition that is selected from the group consisting of cardiac diseases such as heart disease, ischemic infarcts, and cardiometabolic disorders; neurological diseases such as Alzheimer's disease, Parkinson's disease, schizophrenia, and Human Immunodeficiency Virus (HIV) dementia; obesity; metabolic disorders such as insulin related deficiencies and lipid profiles, hepatic diseases, diabetes, and appetite disorders; cancer chemotherapy; benign prostatic hypertrophy; irritable bowel syndrome; biliary diseases; ovarian disorders; marijuana abuse; and alcohol, opioid, nicotine, or cocaine addiction.

In one aspect the diseases include heart disease, ischemic infarcts, cardiometabolic disorders, Alzheimer's disease, Parkinson's disease, schizophrenia, Human Immunodeficiency Virus (HIV), dementia, obesity, hepatic diseases, diabetes, appetite disorders; cancer chemotherapy; benign prostatic hypertrophy; irritable bowel syndrome; biliary diseases; ovarian disorders. In another aspect the disclosed methods can treat diseases of abuse, for example, marijuana abuse, alcohol abuse, opioid abuse, nicotine abuse, or cocaine addiction.

In one aspect, disclosed herein is a method for treating nicotine addiction. The method comprises contacting a subject addicted to nicotine with a composition, comprising:
(a) nicotine infused tobacco leaves;
(b) a bioavailability enhancing agent; and
(c) tobacco leaves.

The infused tobacco leaves of the disclosed methods can further comprise other optional adjunct ingredients as further disclosed herein. Non-limiting examples of which include flavorants, fillers, binders, pH adjusters, buffering agents, colorants, disintegration aids, antioxidants, humectants, and preservatives.

Another aspect of the present disclosure relates methods for treating nicotine addiction comprising contacting a subject in need of treatment with nicotine infused tobacco leaves, comprising:
(a) a therapeutically effective amount of nicotine;
(b) an edible oil; and
(c) tobacco leaves.

As used herein the term “DehydraTECH™” can refer to the final nicotine infused tobacco leaves or by way of reference to the process for preparing all iterations of the disclosed nicotine infused tobacco leaves. In addition, terms such as “pre-DehydraTECH™” and the like refer to intermediate steps in preparing the disclosed nicotine infused tobacco leaves as intermediates prior to the final processing step, i.e., dehydration.

A non-limiting embodiment of this aspect of the present disclosure relates to methods for treating nicotine addiction comprising contacting a subject in need of treatment with nicotine infused tobacco leaves, comprising:
(a) a therapeutically effective amount of nicotine;
(b) an edible oil selected from the group consisting of borage seed oil, syzigium aromaticum oil, hempseed oil, herring oil, cod-liver oil, salmon oil, flaxseed oil, wheat germ oil, evening primrose oils, almond oil, babassu oil, borage oil, black currant seed oil, canola oil, castor oil, coconut oil, corn oil, cottonseed oil, emu oil, evening primrose oil, flax seed oil, grapeseed oil, groundnut oil (e.g., peanut), lanolin oil, linseed oil, mink oil, mustard seed oil, olive oil, palm oil, palm kernel oil, rapeseed oil, safflower oil, sesame oil, shark liver oil, soybean oil, sunflower oil, tree nut oil, hydrogenated castor oil, hydrogenated coconut oil, hydrogenated palm oil, hydrogenated soybean oil, hydrogenated vegetable oil, glyceryl trioleate, glyceryl trilinoleate, glyceryl trilinolenate, citrate thisocetyl triglyceride having 10-18 carbon atoms, omega-3 polyunsaturated fatty acid triglyceride containing oil, omega-3 oil, omega-6 oil, and any combination thereof, and
(c) tobacco leaves;

wherein the nicotine infused tobacco leaves are dehydrated.

In another embodiment the edible oil is selected from the group consisting of canola oil, coconut oil, corn oil, cottonseed oil, olive oil, palm oil, palm kernel oil, rapeseed oil, safflower oil, soybean oil, sunflower oil, hydrogenated coconut oil, hydrogenated palm oil, hydrogenated soybean oil, and any combination thereof.

In one iteration of this aspect of the disclosed methods the nicotine infused tobacco leaves, comprise:
(a) a therapeutically effective amount of nicotine;
(b) an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil; and
(c) tobacco leaves.

In a further aspect nicotine infused tobacco leaves, comprise:
(a) a therapeutically effective amount of nicotine;
(b) an edible oil;
(c) a binder; and
(d) tobacco leaves.

In one iteration of this aspect the nicotine infused tobacco leaves, comprise:
(a) a therapeutically effective amount of nicotine;
(b) an edible oil;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In a further iteration of this aspect the nicotine infused tobacco leaves, comprise:
(a) a therapeutically effective amount of nicotine;
(b) an edible oil an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In one embodiment of this iteration of this aspect, the nicotine infused tobacco leaves, comprise:
(a) a therapeutically effective amount of nicotine;
(b) sunflower oil;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In another embodiment of this iteration of this aspect, the nicotine infused tobacco leaves, comprise:
(a) a therapeutically effective amount of nicotine;
(b) an edible oil an edible oil selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil;
(c) gum arabic; and
(d) tobacco leaves.

A non-limiting example of this embodiment comprises:
(a) a therapeutically effective amount of nicotine;
(b) sunflower oil;
(c) gum arabic; and
(d) tobacco leaves

A still further aspect of the present disclosure relates to methods for treating nicotine addiction comprising contacting a subject in need of treatment with nicotine infused tobacco leaves, comprising:
(a) a therapeutically effective amount of nicotine;
(b) a protective colloid chosen from whole milk, skimmed milk, non-fat dry milk or mixtures thereof; and
(c) tobacco leaves.

A non-limiting embodiment of this aspect of the disclosed nicotine infused tobacco leaves comprise:
(a) a therapeutically effective amount of nicotine;
(b) non-fat dry milk; and
(c) tobacco leaves;

wherein the final nicotine infused tobacco leaves are dehydrated.

The example above, for example, can be prepared by the herein above disclosed DehydraTECH™ process which comprises dehydrating either the final composition or intermediate compositions, wherein the intermediate compositions can be combined with one or more disclosed ingredients which then requires a further dehydration step.

In a further aspect the present disclosure relates to methods for treating nicotine addiction, comprising contacting a subject in need of treatment with nicotine infused tobacco leaves, comprising:
(a) a therapeutically effective amount of nicotine;
(b) a protective colloid;
(c) a binder; and
(d) tobacco leaves.

In one iteration of this aspect the nicotine infused tobacco leaves, comprise:
(a) a therapeutically effective amount of nicotine;
(b) a protective colloid;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In a further iteration of this aspect the nicotine infused tobacco leaves, comprise:
(a) a therapeutically effective amount of nicotine;
(b) a protective colloid chosen from whole milk, skimmed milk, non-fat dry milk or mixtures thereof;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

In one embodiment of this iteration of this aspect, the nicotine infused tobacco leaves, comprise:
(a) a therapeutically effective amount of nicotine;
(b) non-fat dry milk;
(c) a binder selected from the group consisting of povidone, sodium carboxymethylcellulose and other modified cellulosic materials, sodium alginate, xanthan gum, starch-based binders, gum arabic, pectin, carrageenan, pullulan, and zein; and
(d) tobacco leaves.

The active agents of the present disclosure are effective over a wide dosage range. For example, in treating adult humans, compositions and methods of the present disclosure comprise dosages of lipophilic active agents from 0.01 mg to 1,000 mg, from 0.5 mg to 500 mg, from 1 mg to 100 mg, from 5 mg to 50 mg, and from 10 mg to 25 mg. Alternatively, in treating adult humans, compositions and methods of the present disclosure comprise dosages of lipophilic active agents of 0.01 mg, 0.05 mg, 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg.

In one aspect, where the lipophilic active agent within the compositions and methods of the disclosure is a cannabinoid, the condition is selected from the group consisting of cardiac diseases such as heart disease, ischemic infarcts, and cardiometabolic disorders; neurological diseases such as Alzheimer's disease, Parkinson's disease, schizophrenia, and Human Immunodeficiency Virus (HIV) dementia; obesity; metabolic disorders such as insulin related deficiencies and lipid profiles, hepatic diseases, diabetes, and appetite disorders; cancer chemotherapy; benign prostatic hypertrophy; irritable bowel syndrome; biliary diseases; ovarian disorders; marijuana abuse; and alcohol, opioid, nicotine, or cocaine addiction.

In another aspect, where the lipophilic active agent within the compositions and methods of the disclosure is nicotine compound, the condition is a nicotine-related disorder such as tobacco dependence/addiction, Parkinson's disease, ulcerative colitis, Alzheimer's disease, schizophrenia, Attention Deficit Hyperactivity Disorder (ADHD), Tourette's syndrome, ulcerous colitis, and post-smoking-cessation weight control.

In another aspect, where the lipophilic active agent within the compositions and methods of the disclosure is an NSAID as described herein, the condition is pain, fever, and/or an inflammatory-related disease or disorder, including but not limited to asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

In another aspect, where the lipophilic active agent within the compositions and methods of the disclosure is a vitamin, the condition is a vitamin deficiency or condition associated with the lipophilic vitamin. In a particular aspect, where the vitamin is vitamin E as described herein, the condition is vitamin E deficiency and/or a vitamin E related disease or disorder such as ataxia associated with vitamin E deficiency.

In another aspect, a method is provided of treating a central nervous system disease, disorder, or condition, comprising administering any of the compositions disclosed herein to a subject in need thereof.

In other aspects within the methods of treating a central nervous system disease, disorder, or condition in a subject in need thereof, the central nervous system disease, disorder, or condition

(which encompasses psychiatric/behavioral diseases or disorders) may include, but is not limited to, acquired epileptiform aphasia, acute disseminated encephalomyelitis, adrenoleukodystrophy, agenesis of the corpus callosum, agnosia, aicardi syndrome, Alexander disease, Alpers' disease, alternating hemiplegia, Alzheimer's disease, amyotrophic lateral sclerosis, anencephaly, Angelman syndrome, angiomatosis, anoxia, aphasia, apraxia, arachnoid cysts, arachnoiditis, Arnold-chiari malformation, arteriovenous malformation, Asperger's syndrome, ataxia telangiectasia, attention deficit hyperactivity disorder, autism, auditory processing disorder, autonomic dysfunction, back pain, Batten disease, Behcet's disease, Bell's palsy, benign essential blepharospasm, benign focal amyotrophy, benign intracranial hypertension, bilateral frontoparietal polymicrogyria, binswanger's disease, blepharospasm, Bloch-sulzberger syndrome, brachial plexus injury, brain abscess, brain damage, brain injury, brain tumor, spinal tumor, Brown-sequard syndrome, canavan disease, carpal tunnel syndrome (cts), causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, charcot-marie-tooth disease, chiari malformation, chorea, chronic inflammatory demyelinating polyneuropathy (“CIDP”), chronic pain, chronic regional pain syndrome, Coffin lowry syndrome, coma (including persistent vegetative state), congenital facial diplegia, corticobasal degeneration, cranial arteritis, craniosynostosis, Creutzfeldt-jakob disease, cumulative trauma disorders, Cushing's syndrome, cytomegalic inclusion body disease (“CIBD”), cytomegalovirus infection, dandy-walker syndrome, Dawson disease, de morsier's syndrome, Dejerine-klumpke palsy, Dejerine-sottas disease, delayed sleep phase syndrome, dementia, dermatomyositis, developmental dyspraxia, diabetic neuropathy, diffuse sclerosis, dysautonomia, dyscalculia, dysgraphia, dyslexia, dystonia, early infantile epileptic encephalopathy, empty sella syndrome, encephalitis, encephalocele, encephalotrigeminal angiomatosis, encopresis, epilepsy, Erb's palsy, erythromelalgia, essential tremor, Fabry's disease, Fahr's syndrome, fainting, familial spastic paralysis, febrile seizures, fisher syndrome, Friedreich's ataxia, Gaucher's disease, Gerstmann's syndrome, giant cell arteritis, giant cell inclusion disease, globoid cell leukodystrophy, gray matter heterotopia, Guillain-barre syndrome, htlv-1 associated myelopathy, Hallervorden-spatz disease, head injury, headache, hemifacial spasm, hereditary spastic paraplegia, heredopathia atactica polyneuritiformis, herpes zoster oticus, herpes zoster, hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile refsum disease, infantile spasms, inflammatory myopathy, intracranial cyst, intracranial hypertension, Joubert syndrome, Kearns-sayre syndrome, Kennedy disease, kinsbourne syndrome, Klippel feil syndrome, Krabbe disease, Kugelberg-welander disease, kuru, lafora disease, Lambert-eaton myasthenic syndrome, Landau-kleffner syndrome, lateral medullary (Wallenberg) syndrome, learning disabilities, leigh's disease, Lennox-gastaut syndrome, Lesch-nyhan syndrome, leukodystrophy, lewy body dementia, lissencephaly, locked-in syndrome, Lou Gehrig's disease, lumbar disc disease, lyme disease—neurological sequelae, machado-joseph disease (spinocerebellar ataxia type 3), macrencephaly, megalencephaly, Melkersson-rosenthal syndrome, Meniere's disease, meningitis, Menkes disease, metachromatic leukodystrophy, microcephaly, migraine, Miller Fisher syndrome, mini-strokes, mitochondrial myopathies, mobius syndrome, monomelic amyotrophy, motor neurone disease, motor skills disorder, moyamoya disease, mucopolysaccharidoses, multi-infarct dementia, multifocal motor neuropathy, multiple sclerosis, multiple system atrophy with postural hypotension, muscular dystrophy, myalgic encephalomyelitis, myasthenia gravis, myelinoclastic diffuse sclerosis, myoclonic encephalopathy of infants, myoclonus, myopathy, myotubular myopathy, myotonia congenita, narcolepsy, neurofibromatosis, neuroleptic malignant syndrome, neurological manifestations of aids, neurological sequelae of lupus, neuromyotonia, neuronal ceroid lipofuscinosis, neuronal migration disorders, niemann-pick disease, non 24-hour sleep-wake syndrome, nonverbal learning disorder, O'sullivan-mcleod syndrome, occipital neuralgia, occult spinal dysraphism sequence, ohtahara syndrome, olivopontocerebellar atrophy, opsoclonus myoclonus syndrome, optic neuritis, orthostatic hypotension, overuse syndrome, palinopsia, paresthesia, Parkinson's disease, paramyotonia congenita, paraneoplastic diseases, paroxysmal attacks, parry-romberg syndrome (also known as rombergs syndrome), pelizaeus-merzbacher disease, periodic paralyses, peripheral neuropathy, persistent vegetative state, pervasive developmental disorders, photic sneeze reflex, phytanic acid storage disease, pick's disease, pinched nerve, pituitary tumors, pmg, polio, polymicrogyria, polymyositis, porencephaly, post-polio syndrome, postherpetic neuralgia (“PHN”), postinfectious encephalomyelitis, postural hypotension, Prader-willi syndrome, primary lateral sclerosis, prion diseases, progressive hemifacial atrophy (also known as Romberg's syndrome), progressive multifocal leukoencephalopathy, progressive sclerosing poliodystrophy, progressive supranuclear palsy, pseudotumor cerebri, ramsay-hunt syndrome (type I and type II), Rasmussen's encephalitis, reflex sympathetic dystrophy syndrome, refsum disease, repetitive motion disorders, repetitive stress injury, restless legs syndrome, retrovirus-associated myelopathy, rett syndrome, Reye's syndrome, Romberg's syndrome, rabies, Saint Vitus' dance, Sandhoff disease, schizophrenia, Schilder's disease, schizencephaly, sensory integration dysfunction, septo-optic dysplasia, shaken baby syndrome, shingles, Shy-drager syndrome, Sjogren's syndrome, sleep apnea, sleeping sickness, snatiation, Sotos syndrome, spasticity, spina bifida, spinal cord injury, spinal cord tumors, spinal muscular atrophy, spinal stenosis, Steele-richardson-olszewski syndrome, see progressive supranuclear palsy, spinocerebellar ataxia, stiff-person syndrome, stroke, Sturge-weber syndrome, subacute sclerosing panencephalitis, subcortical arteriosclerotic encephalopathy, superficial siderosis, sydenham's chorea, syncope, synesthesia, syringomyelia, tardive dyskinesia, Tay-sachs disease, temporal arteritis, tetanus, tethered spinal cord syndrome, Thomsen disease, thoracic outlet syndrome, tic douloureux, Todd's paralysis, Tourette syndrome, transient ischemic attack, transmissible spongiform encephalopathies, transverse myelitis, traumatic brain injury, tremor, trigeminal neuralgia, tropical spastic paraparesis, trypanosomiasis, tuberous sclerosis, vasculitis including temporal arteritis, Von Hippel-lindau disease (“VHL”), Viliuisk encephalomyelitis (“VE”), Wallenberg's syndrome, Werdnig-hoffman disease, west syndrome, whiplash, Williams syndrome, Wilson's disease, and Zellweger syndrome. It is thus appreciated that all CNS-related states and disorders could be treated through the BBB route of drug delivery.

In some embodiments, a CNS disease, disorder, or condition according to embodiments of the present disclosure may be selected from a metabolic disease, a behavioral disorder, a personality disorder, dementia, a cancer, a neurodegenerative disorder, pain, a viral infection, a sleep disorder, a seizure disorder, acid lipase disease, Fabry disease, Wernicke-Korsakoff syndrome, ADHD, anxiety disorder, borderline personality disorder, bipolar disorder, depression, eating disorder, obsessive-compulsive disorder, schizophrenia, Alzheimer's disease, Barth syndrome and Tourette's syndrome, Canavan disease, Hallervorden-Spatz disease, Huntington's disease, Lewy Body disease, Lou Gehrig's disease, Machado-Joseph disease, Parkinson's disease, or Restless Leg syndrome. In some embodiments, the CNS disease, disorder, or condition is pain and is selected from neuropathic pain, central pain syndrome, somatic pain, visceral pain, and/or headache.

As used herein, the term “subject” treated by the presently disclosed methods in their many aspects is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or the prophylactic diagnosis or treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some aspects, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular composition employed, the route of administration, the time of administration, the rate of excretion of the particular composition being employed, the duration of the treatment, other drugs, and/or materials used in combination with the particular composition employed, the age, sex, weight, condition, general health and prior medical history of the patient being treated, and like factors well known in the medical arts.

A physician having ordinary skill in the art can readily determine and prescribe the effective amount of the presently disclosed composition required. Accordingly, the dosage range for administration may be adjusted by the physician as necessary, as described more fully elsewhere herein.

### EXAMPLES

### Example 1

A line of CBD and/or THC infused tea bags coming in a variety of flavors was developed.

### I. Ingredients:

Tea in leaf form, oil form, brewed form, organic and inorganic;

Evaporated dry non-fat milk;

CBD oil;

Hemp oil or compatible oil for ingestion;

Cannabis leaves, buds, oils; all strains with THC and/or CBD.

### II. ViPova® Formulas:

IIA. CBD Tea
   Combine evaporated nonfat, dry milk with any and all teas, organic and inorganic;
   Blend CBD oil with the tea leaves;
   Dehydrate mixture of tea, CBD oil, and evaporated nonfat dry milk in a food dehydrator;
   End-product is ViPova® Tea with CBD enhancement only.
IIB. THC/CBD Tea
   Combine evaporated nonfat, dry milk with any and all teas, organic and inorganic;
   Blend hemp or other ingestible oil with the tea leaves;
   Add cannabis leaves to above mixture;
   Dehydrate mixture of tea, hemp or other ingestible oil, cannabis leaves, and evaporated nonfat dry milk;
   End-product is ViPova® Tea with THC and CBD.

### III. ViPova® Formulas: Specifications:

IIIA. CBD Tea
   Tea: one tea bag contains 1 gram to 3 grams of tea leaves (dry weight);
   Evaporated dry non-fat milk: 0.10-1.00 grams;
   CBD oil: 10 mgs.-25 mgs. per tea bag;
IIIB. THC/CBD Tea
   Tea: one tea bag contains 1.5-12 grams tea leaves (dry weight) per tea bag;
   Evaporated dry milk: 0.10-6.00 grams per tea bag;
   Hemp oil or other ingestible oil: 10 mgs.-25 mgs. per tea bag;
   Cannabis leaves: 1.00-12.00 grams per tea bag;
IIIC. Production Equipment:
   Commercial grinder for tea and/or cannabis leaves;
   Commercial mixer;
   Commercial dehydrator;
   Commercial tea bag filling machine.

### IV. Flavorings

ViPova® Teas will provide a menu of flavorings for addition to tea bags or loose tea selections including, but not limited to mint, citrus, and vanilla.

### Example 2

A process for adhering CBD and/or THC to food products was developed. The food products may be selected from the group consisting of meats, fish, fruits, vegetables, dairy products, legumes, pastas, breads, grains, seeds, nuts, spices, and herbs. The process may or may not involve contacting the food product with sunflower and/or dry evaporated milk. The process involved the steps of:
1. A food product was saturated with 0-60 grams of CBD and/or THC oil or extract.
2. The food product was placed on dehydrator paper and placed in a food dehydrator for 0-24 hours.
3. The food product was removed from the dehydrator and stored in air-tight containers.

### Example 3

Black tea was formulated with various lipophilic active agents. Active agents were dosed into the tea at a concentration of approximately 4.5 mg of active ingredient per gram of finished product, using non-fat dry milk and sunflower seed oil as excipients. The following ingredients were used for the formulation:

453 g of loose leaf black tea;

2265 mg active agent;

45 g of instant non-fat dry evaporated milk;

1132.5 mg of sunflower seed oil;

Ingredients were combined in a stainless steel bowl and mixed with gloved hands. A homogenous mixture was spread evenly on a dehydrator tray and dehydrated for 30 minutes.

After cooling, the formulated tea was placed into a sterile zip-lock bag. The active ingredients that were formulated were: ASA (aspirin), ibuprofen, acetaminophen, diclofenac, indomethacin, piroxicam, nicotine, and vitamin E (α-tocopherol). The specific supplier information and lot numbers for each active agent are shown below in Table 1.

TABLE 1
Active Agents Used for Formulations

| | CAS | | Catalogue | |
|---|---|---|---|---|
| Compound | Number | Supplier | Number | Lot Number |
| ASA (aspirin) | 50-78-2 | Sigma-Aldrich | A2093 | #MKBQ8444V |
| Ibuprofen | 15687-27-1 | Sigma-Aldrich | I4883 | #MKBQ4505V |
| Acetaminophen | 103-90-2 | Sigma-Aldrich | A5000 | #MKBS7142V |
| Diclofenac | 15307-79-6 | Sigma-Aldrich | D6899 | #BCBN3367V |
| Indomethacin | 53-86-1 | Sigma-Aldrich | I8280 | #MKBR4530V |
| Piroxicam | 36322-90-4 | Sigma-Aldrich | P0847 | #SLBF3478V |
| Nicotine | 54-11-5 | Sigma-Aldrich | N3876 | #1449194V |
| Vitamin E | 10191-41-0 | Sigma-Aldrich | 258024 | #MKBT5983V |
| (α-tocopherol) | | | | |

The Tea used was loose leaf English Breakfast Tea from Upton Tea Imports (Holliston, Mass.).

The Sunflower Oil was Whole Foods brand organic sunflower oil.

The non-fat dry milk power was NowFoods brand organic non-fat dry milk.

The dehydrator used was a Presto Dehydrator, model #06300.

Each component of the formulation was weighed out and combined as described in the above procedure. The weights of the individual active agents for each formulation are summarized below in Table 2.

TABLE 2
Formulation of Active Agents

| | Compound | Non-Fat | Sunflower | Black | | Compound |
|---|---|---|---|---|---|---|
| Compound | Weight | Dry Milk | Seed Oil | Tea | Yield | Concentration |
| ASA (aspirin) | 2267.1 mg | 45.09 g | 1135 mg | 453.2 g | 479.3 g | 4.52 mg/g |
| Ibuprofen | 2265.5 mg | 45.05 g | 1138 mg | 453.8 g | 488.1 g | 4.51 mg/g |
| Acetaminophen | 2264.7 mg | 45.01 g | 1136 mg | 453.2 g | 477.9 g | 4.51 mg/g |
| Diclofenac | 2265.3 mg | 45.06 g | 1133 mg | 453.1 g | 441.3 g | 4.52 mg/g |
| Indomethacin | 2266.3 mg | 44.99 g | 1138 mg | 453.1 g | 491.5 g | 4.52 mg/g |
| Piroxicam | 2265.9 mg | 45.25 g | 1134 mg | 453.6 g | 488.3 g | 4.51 mg/g |
| Nicotine | 2264.9 mg | 45.02 g | 1133 mg | 453.1 g | 488.1 g | 4.52 mg/g |
| Vitamin E | 2271.1 mg | 45.05 g | 1135 mg | 453.2 g | 480.2 g | 4.53 mg/g |
| (α-tocopherol) | | | | | | |

For each formulation, the constituents were mixed by hand until a homogeneous mixture was achieved, then spread evenly on dehydrator trays for drying. Each formulation was dried for 30 minutes in dehydrator. After cooling, mixture was placed into Zip-Lock bag. After taring the analytical balance for the Zip-Lock bag, the weight of the final formulation was recorded and the concentration of active ingredients in the formulation calculated (Table 2).

### Example 4

As used herein, compositions incorporating DehydraTECH™ are compositions that incorporate a dehydrated mixture comprising a therapeutically effective amount of a lipophilic active agent and an edible oil comprising long chain fatty acids, particularly wherein dehydrated mixture is obtainable by the steps of
i) combining a therapeutically effective amount of the lipophilic active agent with the edible oil comprising long chain fatty acids; and
ii) dehydrating the product of step (i), thereby producing the dehydrated mixture.

The following test compositions were used in the animal studies disclosed in Example 4. Nicotine polacrilex (12.5 g) and sunflower oil (12.5 g) were combined and intimately mixed to form a lipophilic active agent (nicotine polacrilex) and edible oil admixture. The edible oil admixture was then combined with gum Arabic (25 g) and thoroughly mixed to form a pre-DehydraTECH™ composition. The pre-DehydraTECH™ composition is then dehydrated to form a flowable powder that can be administered to the test animals orally as an aqueous suspension. The control composition is an aqueous suspension of nicotine polacrilex given in a sufficient amount as described herein below.

The nicotine polacrilex used in Example 4 is a 20% nicotine composition wherein the nicotine is bound to a polyhmethacrylic acid ion-exchange resin, for example, Amberlite RP64, Purolite C115HMR or Doshion P551.

This study was designed to principally assess the relative ingestible nicotine absorption performance of DehydraTECH™_powered formulations compared to concentration-matched control formulations that lacked any form of delivery enabling technology in rats. Nicotine was administered in a nicotine polacrilex derivative format as is widely commercialized today in nicotine replacement therapy products such as chewing gums. Twelve male rats were divided into four groups of three, such that DehydraTECH™ and control formulations were each tested at a 1 mg/Kg and 10 mg/Kg dosage level. Formulations were administered orally and all rats were cannulated for blood collection at multiple intervals over an 8 hour duration post-dosing with the first data collection at the 15-minute mark. Urine and feces were also collected for up to a 24-hour duration post-dosing, and essential organ tissue samples were also collected for examination after the study. All samples were subjected to analytical testing in order to quantify the levels of nicotine therein, as well as the levels of three major liver metabolites thereof, hydroxycotinine, nicotine N′-oxide and cotinine, in order to assess the relative metabolite levels absorbed by the different formulations.

### Results & Observations

The DehydraTECH™ formulations generally achieved faster absorption, higher peak absorption and higher overall quantities of nicotine, on average, in the blood than the concentration-matched control formulations at both the 1 mg and 10 mg/Kg doses tested. Furthermore, as previously reported, there were no obvious signs of gastrointestinal distress such as vomiting or diarrhea indicating that the animals appeared to tolerate the treatment well.

Nicotine blood levels were evaluated multiple times over a period of 8 hours after dosing. In the 10 mg/Kg dosing arm, the control formulation required nearly 3 hours to reach similar levels of blood absorption that the DehydraTECH™ formulation reached in only 15 minutes. Furthermore, the DehydraTECH™ formulation went on thereafter to demonstrate peak plasma levels that were 148% of those achieved by the control formulation. If replicated in human studies, these findings are suggestive that DehydraTECH™'s technology could prove more effective in elevating blood nicotine levels through edible formats much more quickly and substantially than previously theorized, potentially making ingestible nicotine preparations a viable alternative to today's available product formats while also leading to a more rapid nicotine craving satiation.

Analysis of the liver metabolites revealed, as expected, that overall levels in the blood of two of the three metabolites studied were higher in the control group than in the DehydraTECH™ formulation group at the 10 mg/Kg dose. This result was especially pronounced in the 45-minute to 2-hour time interval post-dosing which is consistent with the expected timing of release of metabolites in higher quantity into the bloodstream by the liver following normal physiological processing of ingested nicotine with the control preparation, compared to the DehydraTECH™ technology that is believed to elude first pass liver metabolism. The DehydraTECH™ formulation also demonstrated lower quantities of nicotine in the rat urine at both doses, which is consistent with the fact that the levels of nicotine in the rat blood remained higher over the duration of the study with the DehydraTECH™ formulation than with the control. The study also revealed that the DehydraTECH™ formulation at the 10 mg/Kg level achieved up to 5.6-times as much nicotine upon analysis of the rat brain tissue than was recovered with the matching control formulation. These findings together perhaps suggest prolongation of nicotine effectiveness with the DehydraTECH™ formulation which may also be beneficial in humans to control cravings over an extended time-period from a single edible nicotine dose.

### Example 5

In this study, the exposure and distribution of nicotine and its major metabolites were evaluated following oral administration of two separate formulations (Reference and Test Nicotine Polacrilex) in male Sprague-Dawley rats.

The following test compositions were used in the animal studies disclosed in Example 5. Nicotine polacrilex (12.5 g) and sunflower oil (12.5 g) were combined and intimately mixed to form a lipophilic active agent (nicotine polacrilex) and edible oil admixture. The edible oil admixture was then combined with gum Arabic (25 g) and thoroughly mixed to form a pre-DehydraTECH™ composition. The pre-DehydraTECH™ composition is then dehydrated to form a flowable powder that can be administered to the test animals orally as an aqueous suspension. The control composition is a aqueous suspension of nicotine polacrilex given in a sufficient amount as described herein below.

The nicotine polacrilex used in Example 5 is a 20% nicotine composition wherein the nicotine is bound to a polyhmethacrylic acid ion-exchange resin, for example, Amberlite 1RP64, Purolite C115HMR or Doshion P551.

Formulations were administered orally (PO) at 10 mg/kg. Following dosing, blood samples were collected up to 1 hour post dose; and urine and fecal samples were collected up to 24 hours post dose. Brain, liver, and kidney tissue were collected at 1 hour (Groups 1 & 5), 4 hours (Groups 2 & 6), following the 8 hour urine and feces sample collection (Groups 3 & 7), or following the 24 hour urine and feces sample collection (Groups 4 & 8). Blood, urine, feces, and tissue concentrations of each analyte were determined by LC-MS/MS. Plasma pharmacokinetic parameters were determined using WinNonlin (v8.0). Brain, liver, and kidney pharmacokinetic parameters were determined using WinNonlin (v8.0) software with sparse sampling.

Following PO dosing of Reference Nicotine Polacrilex at 10 mg/kg (Group 1), maximum plasma concentrations (average of 144±68.2 ng/mL) of nicotine were observed between 30 minutes and 1 hour post dosing. The average half-life after oral dosing could not be determined. The average exposure for nicotine (Group 1) based on the dose normalized AUClast was 8.71±2.76 hr*kg*ng/mL/mg.

Following PO dosing of Reference Nicotine Polacrilex at 10 mg/kg (Group 1), maximum plasma concentrations (average of 9.79±3.56 ng/mL) of hydroxycotinine metabolite were observed between 45 minutes and 1 hour post dosing. The average half-life after oral dosing could not be determined. The average exposure for hydroxycotinine (Group 1) based on the dose normalized AUClast was 0.420±0.146 hr*kg*ng/mL/mg.

Following PO dosing of Reference Nicotine Polacrilex at 10 mg/kg (Group 1), maximum plasma concentrations (average of 179±54.9 ng/mL) of nicotine-n-oxide metabolite were observed between 30 minutes and 1 hour post dosing. The average half-life after oral dosing could not be determined. The average exposure for nicotine-n-oxide (Group 1) based on the dose normalized AUClast was 11.2±3.32 hr*kg*ng/mL/mg.

Following PO dosing of Reference Nicotine Polacrilex at 10 mg/kg (Group 1), maximum plasma concentrations (average of 193±58.6 ng/mL) of cotinine metabolite were observed at 1 hour post dosing. The average half-life after oral dosing could not be determined. The average exposure for cotinine (Group 1) based on the dose normalized AUClast was 10.9±2.90 hr*kg*ng/mL/mg.

Following PO dosing of Reference Nicotine Polacrilex at 10 mg/kg (Group 2), maximum plasma concentrations (average of 350±256 ng/mL) of nicotine were observed between 8 minutes and 1 hour post dosing. The average half-life after oral dosing could not be determined. The average exposure for nicotine (Group 2) based on the dose normalized AUClast was 21.3±13.7 hr*kg*ng/mL/mg.

Following PO dosing of Reference Nicotine Polacrilex at 10 mg/kg (Group 2), maximum plasma concentrations (average of 20.1±13.3 ng/mL) of hydroxycotinine metabolite were observed at 1 hour post dosing. The average half-life after oral dosing could not be determined. The average exposure for hydroxycotinine (Group 2) based on the dose normalized AUClast was 1.15±0.928 hr*kg*ng/mL/mg.

Following PO dosing of Reference Nicotine Polacrilex at 10 mg/kg (Group 2), maximum plasma concentrations (average of 409±235 ng/mL) of nicotine-n-oxide metabolite were observed between 12 minutes and 1 hour post dosing. The average half-life after oral dosing could not be determined. The average exposure for nicotine-n-oxide (Group 2) based on the dose normalized AUClast was 26.8±18.3 hr*kg*ng/mL/mg.

Following PO dosing of Reference Nicotine Polacrilex at 10 mg/kg (Group 2), maximum plasma concentrations (average of 359±236 ng/mL) of cotinine metabolite were observed between 45 minutes and 1 hour post dosing. The average half-life after oral dosing could not be determined. The average exposure for cotinine (Group 2) based on the dose normalized AUClast was 22.5±16.7 hr*kg*ng/mL/mg.

Following PO dosing of Reference Nicotine Polacrilex at 10 mg/kg (Group 3), maximum plasma concentrations (average of 176±71.2 ng/mL) of nicotine were observed between 30 minutes and 1 hour post dosing. The average half-life after oral dosing could not be determined. The average exposure for nicotine (Group 3) based on the dose normalized AUClast was 11.7±4.62 hr*kg*ng/mL/mg. On average, 1.04±0.49% and 0.03±0.04% of the dose (unchanged dose) was found in urine and feces, respectively, after PO dosing.

Following PO dosing of Reference Nicotine Polacrilex at 10 mg/kg (Group 3), maximum plasma concentrations (average of 13.4±5.95 ng/mL) of hydroxycotinine metabolite were observed between 45 minutes 1 hour post dosing. The average half-life after oral dosing could not be determined. The average exposure for hydroxycotinine (Group 3) based on the dose normalized AUClast was 0.672±0.386 hr*kg*ng/mL/mg. On average, 1.10±0.64% and 0.03% (n=1) of the dose was found in urine and feces, respectively, after PO dosing.

Following PO dosing of Reference Nicotine Polacrilex at 10 mg/kg (Group 3), maximum plasma concentrations (average of 283±134 ng/mL) of nicotine-n-oxide metabolite were observed between 30 minutes and 1 hour post dosing. The average half-life after oral dosing could not be determined. The average exposure for nicotine-n-oxide (Group 3) based on the dose normalized AUClast was 17.8±7.29 hr*kg*ng/mL/mg. On average, 9.36±4.36% and 0.07% (n=1) of the dose was found in urine and feces, respectively, after PO dosing.

Following PO dosing of Reference Nicotine Polacrilex at 10 mg/kg (Group 3), maximum plasma concentrations (average of 304±103 ng/mL) of cotinine metabolite were observed at 1 hour post dosing. The average half-life after oral dosing could not be determined. The average exposure for cotinine (Group 3) based on the dose normalized AUClast was 15.4±4.99 hr*kg*ng/mL/mg. On average, 0.99±0.48% and 0.03±0.02% of the dose was found in urine and feces, respectively, after PO dosing.

Following PO dosing of Reference Nicotine Polacrilex at 10 mg/kg (Group 4), maximum plasma concentrations (average of 210±68.6 ng/mL) of nicotine were observed between 15 minutes and 1 hour post dosing. The average half-life after oral dosing was 0.949±0.214 hours. The average exposure for nicotine (Group 4) based on the dose normalized AUClast was 13.0±4.98 hr*kg*ng/mL/mg. On average, 3.31±0.91% and 0.09±0.07% of the dose (unchanged dose) was found in urine and feces, respectively, after PO dosing.

Following PO dosing of Reference Nicotine Polacrilex at 10 mg/kg (Group 4), maximum plasma concentrations (average of 14.3±4.74 ng/mL) of hydroxycotinine metabolite were observed between 45 minutes and 1 hour post dosing. The average half-life after oral dosing could not be determined. The average exposure for hydroxycotinine (Group 4) based on the dose normalized AUClast was 0.751±0.389 hr*kg*ng/mL/mg. On average, 6.48±2.12% and 0.03±0.02% of the dose was found in urine and feces, respectively, after PO dosing.

Following PO dosing of Reference Nicotine Polacrilex at 10 mg/kg (Group 4), maximum plasma concentrations (average of 223±71.9 ng/mL) of nicotine-n-oxide metabolite were observed between 15 minutes and 1 hour post dosing. The average half-life after oral dosing was 1.38 hours. The average exposure for nicotine-n-oxide (Group 4) based on the dose normalized AUClast was 15.0±6.27 hr*kg*ng/mL/mg. On average, 20.3±6.90% of the dose was found in urine after PO dosing. All concentrations in feces were below the limit of quantitation.

Following PO dosing of Reference Nicotine Polacrilex at 10 mg/kg (Group 4), maximum plasma concentrations (average of 247±49.4 ng/mL) of cotinine metabolite were observed between 45 minutes and 1 hour post dosing. The average half-life after oral dosing could not be determined. The average exposure for cotinine (Group 4) based on the dose normalized AUClast was 14.0±2.60 hr*kg*ng/mL/mg. On average, 5.30±2.18% and 0.16±0.08% of the dose was found in urine and feces, respectively, after PO dosing.

Following PO dosing of Reference Nicotine Polacrilex at 10 mg/kg (Groups 1-4), the average (±SE) Cₘₐₓ for nicotine in brain tissue was 427±66.5 ng/g, the tₘₐₓ was 4 hours, the half-life could not be determined, and the exposure for nicotine based on the dose normalized AUClast was 588±53.8 hr*kg*ng/g/mg. After PO dosing of Reference Nicotine Polacrilex, the average (±SE) Cₘₐₓ for hydroxycotinine metabolite in brain tissue was 51.8±9.14 ng/g, the tₘₐₓ was 8 hours, the half-life could not be determined, and the exposure for hydroxycotinine metabolite based on the dose normalized AUClast was 95.5±12.1 hr*kg*ng/g/mg. After PO dosing of Reference Nicotine Polacrilex, the majority of the concentrations were below the limit of quantitation and therefore, the pharmacokinetic parameters were not able to be calculated. After PO dosing of Reference Nicotine Polacrilex, the average (±SE) Cₘₐₓ for cotinine metabolite in brain tissue was 722±135 ng/g, the tₘₐₓ was 8 hours, the half-life could not be determined, and the exposure for cotinine metabolite based on the dose normalized AUClast was 1332±208 hr*kg*ng/g/mg.

Following PO dosing of Reference Nicotine Polacrilex at 10 mg/kg (Groups 1-4), the average (±SE) Cₘₐₓ for nicotine in liver tissue was 1300±308 ng/g, the tₘₐₓ was 4 hours, the half-life could not be determined, and the exposure for nicotine based on the dose normalized AUClast was 1737±167 hr*kg*ng/g/mg. After PO dosing of Reference Nicotine Polacrilex, the average (±SE) Cₘₐₓ for hydroxycotinine metabolite in liver tissue was 102±13.5 ng/g, the tₘₐₓ was 8 hours, the half-life could not be determined, and the exposure for hydroxycotinine metabolite based on the dose normalized AUClast was 205±26.3 hr*kg*ng/g/mg. After PO dosing of Reference Nicotine Polacrilex, the average (±SE) Cₘₐₓ for nicotine-n-oxide metabolite in liver tissue was 4.51±1.58 ng/g, the tₘₐₓ was 8 hours, the half-life could not be determined, and the exposure for nicotine-n-oxide metabolite based on the dose normalized AUClast was 6.86±1.83 hr*kg*ng/g/mg. After PO dosing of Reference Nicotine Polacrilex, the average (±SE) Cₘₐₓ for cotinine metabolite in liver tissue was 905±119 ng/g, the tₘₐₓ was 8 hours, the half-life could not be determined, and the exposure for cotinine metabolite based on the dose normalized AUClast was 1620±189 hr*kg*ng/g/mg.

Following PO dosing of Reference Nicotine Polacrilex at 10 mg/kg (Groups 1-4), the average (±SE) Cₘₐₓ for nicotine in kidney tissue was 8965±1519 ng/g, the tₘₐₓ was 4 hours, the half-life could not be determined, and the exposure for nicotine based on the dose normalized AUClast was 12267±1173 hr*kg*ng/g/mg. After PO dosing of Reference Nicotine Polacrilex, the average (±SE) Cₘₐₓ for hydroxycotinine metabolite in kidney tissue was 200±44.1 ng/g, the tₘₐₓ was 24 hours, the half-life could not be determined, and the exposure for hydroxycotinine metabolite based on the dose normalized AUClast was 391±47.7 hr*kg*ng/g/mg. After PO dosing of Reference Nicotine Polacrilex, the average (SE) Cₘₐₓ for nicotine-n-oxide metabolite in kidney tissue was 20.5±4.26 ng/g, the tₘₐₓ was 4 hours, the half-life could not be determined, and the exposure for nicotine-n-oxide metabolite based on the dose normalized AUClast was 23.4±2.80 hr*kg*ng/g/mg. After PO dosing of Reference Nicotine Polacrilex, the average (±SE) Cₘₐₓ for cotinine metabolite in kidney tissue was 1775±217 ng/g, the tₘₐₓ was 8 hours, the half-life could not be determined, and the exposure for cotinine metabolite based on the dose normalized AUClast was 3436±374 hr*kg*ng/g/mg.

Following PO dosing of Test Nicotine Polacrilex at 10 mg/kg (Group 5), maximum plasma concentrations (average of 416±255 ng/mL) of nicotine were observed between 12 minutes and 1 hour post dosing. The average half-life after oral dosing could not be determined. The average exposure for nicotine (Group 5) based on the dose normalized AUClast was 28.7±13.8 hr*kg*ng/mL/mg.

Following PO dosing of Test Nicotine Polacrilex at 10 mg/kg (Group 5), maximum plasma concentrations (average of 13.9±3.07 ng/mL) of hydroxycotinine metabolite were observed at 1 hour post dosing. The average half-life after oral dosing could not be determined. The average exposure for hydroxycotinine (Group 5) based on the dose normalized AUClast was 0.671±0.167 hr*kg*ng/mL/mg.

Following PO dosing of Test Nicotine Polacrilex at 10 mg/kg (Group 5), maximum plasma concentrations (average of 267±56.1 ng/mL) of nicotine-n-oxide metabolite were observed between 45 minutes and 1 hour post dosing. The average half-life after oral dosing could not be determined. The average exposure for nicotine-n-oxide (Group 5) based on the dose normalized AUClast was 19.3±3.45 hr*kg*ng/mL/mg.

Following PO dosing of Test Nicotine Polacrilex at 10 mg/kg (Group 5), maximum plasma concentrations (average of 381±81.8 ng/mL) of cotinine metabolite were observed at 1 hour post dosing. The average half-life after oral dosing could not be determined. The average exposure for cotinine (Group 5) based on the dose normalized AUClast was 21.3±5.76 hr*kg*ng/mL/mg.

Following PO dosing of Test Nicotine Polacrilex at 10 mg/kg (Group 6), maximum plasma concentrations (average of 315±142 ng/mL) of nicotine were observed between 15 minutes and 1 hour post dosing. The average half-life after oral dosing could not be determined. The average exposure for nicotine (Group 6) based on the dose normalized AUClast was 21.5±10.8 hr*kg*ng/mL/mg.

Following PO dosing of Test Nicotine Polacrilex at 10 mg/kg (Group 6), maximum plasma concentrations (average of 11.6±2.62 ng/mL) of hydroxycotinine metabolite were observed between 45 minutes and 1 hour post dosing. The average half-life after oral dosing could not be determined. The average exposure for hydroxycotinine (Group 6) based on the dose normalized AUClast was 0.581±0.149 hr*kg*ng/mL/mg.

Following PO dosing of Test Nicotine Polacrilex at 10 mg/kg (Group 6), maximum plasma concentrations (average of 246±120 ng/mL) of nicotine-n-oxide metabolite were observed between 15 minutes and 1 hour post dosing. The average half-life after oral dosing could not be determined. The average exposure for nicotine-n-oxide (Group 6) based on the dose normalized AUClast was 15.6±8.37 hr*kg*ng/mL/mg.

Following PO dosing of Test Nicotine Polacrilex at 10 mg/kg (Group 6), maximum plasma concentrations (average of 315±76.8 ng/mL) of cotinine metabolite were observed between 45 minutes and 1 hour post dosing. The average half-life after oral dosing could not be determined. The average exposure for cotinine (Group 6) based on the dose normalized AUClast was 17.7±5.25 hr*kg*ng/mL/mg.

Following PO dosing of Test Nicotine Polacrilex at 10 mg/kg (Group 7), maximum plasma concentrations (average of 253±40.0 ng/mL) of nicotine were observed between 12 minutes and 1 hour post dosing. The average half-life after oral dosing could not be determined. The average exposure for nicotine (Group 7) based on the dose normalized AUClast was 18.3±6.21 hr*kg*ng/mL/mg. On average, 2.02±1.21% and 0.04±0.04% of the dose (unchanged dose) was found in urine and feces, respectively, after PO dosing.

Following PO dosing of Test Nicotine Polacrilex at 10 mg/kg (Group 7), maximum plasma concentrations (average of 12.7±4.62 ng/mL) of hydroxycotinine metabolite were observed at 1 hour post dosing. The average half-life after oral dosing could not be determined. The average exposure for hydroxycotinine (Group 7) based on the dose normalized AUClast was 0.620±0.253 hr*kg*ng/mL/mg. On average, 0.97±0.34% and 0.02% (n=1) of the dose was found in urine and feces, respectively, after PO dosing.

Following PO dosing of Test Nicotine Polacrilex at 10 mg/kg (Group 7), maximum plasma concentrations (average of 276±67.5 ng/mL) of nicotine-n-oxide metabolite were observed between 15 minutes and 1 hour post dosing. The average half-life after oral dosing was 2.84 hours. The average exposure for nicotine-n-oxide (Group 7) based on the dose normalized AUClast was 17.6±6.17 hr*kg*ng/mL/mg. On average, 9.91±4.61% and 0.12% of the dose was found in urine and feces, respectively, after PO dosing.

Following PO dosing of Test Nicotine Polacrilex at 10 mg/kg (Group 7), maximum plasma concentrations (average of 317±100 ng/mL) of cotinine metabolite were observed at 1 hour post dosing. The average half-life after oral dosing could not be determined. The average exposure for cotinine (Group 7) based on the dose normalized AUClast was 16.6±4.69 hr*kg*ng/mL/mg. On average, 1.39±0.80% and 0.02±0.01% of the dose was found in urine and feces, respectively, after PO dosing.

Following PO dosing of Test Nicotine Polacrilex at 10 mg/kg (Group 8), maximum plasma concentrations (average of 593±641 ng/mL) of nicotine were observed between 8 minutes and 1 hour post dosing. The average half-life after oral dosing could not be determined; however, the half-life for one rat was 0.737 hours. The average exposure for nicotine (Group 8) based on the dose normalized AUClast was 38.0±38.5 hr*kg*ng/mL/mg. On average, 5.91±3.24% and 0.06±0.03% of the dose (unchanged dose) was found in urine and feces, respectively, after PO dosing.

Following PO dosing of Test Nicotine Polacrilex at 10 mg/kg (Group 8), maximum plasma concentrations (average of 17.4±13.8 ng/mL) of hydroxycotinine metabolite were observed between 45 minutes and 1 hour post dosing. The average half-life after oral dosing could not be determined. The average exposure for hydroxycotinine (Group 8) based on the dose normalized AUClast was 0.940±0.788 hr*kg*ng/mL/mg. On average, 9.07±3.61% and 0.02±0.01% of the dose was found in urine and feces, respectively, after PO dosing.

Following PO dosing of Test Nicotine Polacrilex at 10 mg/kg (Group 8), maximum plasma concentrations (average of 357±306 ng/mL) of nicotine-n-oxide metabolite were observed between 15 minutes and 1 hour post dosing. The average half-life after oral dosing could not be determined; however, the half-life for one rat was 0.888 hours. The average exposure for nicotine-n-oxide (Group 8) based on the dose normalized AUClast was 27.5±23.8 hr*kg*ng/mL/mg. On average, 39.5±9.71% and 0.08% of the dose was found in urine and feces, respectively, after PO dosing.

Following PO dosing of Test Nicotine Polacrilex at 10 mg/kg (Group 8), maximum plasma concentrations (average of 441±333 ng/mL) of cotinine metabolite were observed at 1 hour post dosing. The average half-life after oral dosing could not be determined. The average exposure for cotinine (Group 8) based on the dose normalized AUClast was 25.8±20.0 hr*kg*ng/mL/mg. On average, 8.23±2.58% and 0.18±0.10% of the dose was found in urine and feces, respectively, after PO dosing.

Following PO dosing of Test Nicotine Polacrilex at 10 mg/kg (Groups 5-8), the average (±SE) Cₘₐₓ for nicotine in brain tissue was 1260±200 ng/g, the tₘₐₓ was 1 hour, the half-life was 21.6 hours, and the exposure for nicotine based on the dose normalized AUClast was 1300±125 hr*kg*ng/g/mg. After PO dosing of Test Nicotine Polacrilex, the average (±SE) Cₘₐₓ for hydroxycotinine metabolite in brain tissue was 91.2±7.69 ng/g, the tₘₐₓ was 24 hours, the half-life could not be determined, and the exposure for hydroxycotinine metabolite based on the dose normalized AUClast was 142±6.64 hr*kg*ng/g/mg. After PO dosing of Test Nicotine Polacrilex, the average (±SE) Cₘₐₓ for nicotine-n-oxide metabolite in brain tissue was 4.17±1.41 ng/g, the tₘₐₓ was 1 hour, the half-life could not be determined, and the exposure for nicotine-n-oxide metabolite based on the dose normalized AUClast was 2.70±1.05 hr*kg*ng/g/mg. After PO dosing of Test Nicotine Polacrilex, the average (±SE) Cₘₐₓ for cotinine metabolite in brain tissue was 1322±219 ng/g, the tₘₐₓ was 24 hours, the half-life could not be determined, and the exposure for cotinine metabolite based on the dose normalized AUClast was 2172±189 hr*kg*ng/g/mg.

Following PO dosing of Test Nicotine Polacrilex at 10 mg/kg (Groups 5-8), the average (±SE) Cₘₐₓ for nicotine in liver tissue was 2702±308 ng/g, the tₘₐₓ was 1 hour, the half-life was 18.9 hours, and the exposure for nicotine based on the dose normalized AUClast was 2989±277 hr*kg*ng/g/mg. After PO dosing of Test Nicotine Polacrilex, the average (±SE) Cₘₐₓ for hydroxycotinine metabolite in liver tissue was 232±41.2 ng/g, the tₘₐₓ was 24 hours, the half-life could not be determined, and the exposure for hydroxycotinine metabolite based on the dose normalized AUClast was 338±37.6 hr*kg*ng/g/mg. After PO dosing of Test Nicotine Polacrilex, the average (±SE) Cₘₐₓ for nicotine-n-oxide metabolite in liver tissue was 6.69±1.67 ng/g, the tₘₐₓ was 1 hour, the half-life could not be determined, and the exposure for nicotine-n-oxide metabolite based on the dose normalized AUClast was 8.74±2.56 hr*kg*ng/g/mg. After PO dosing of Test Nicotine Polacrilex, the average (±SE) Cₘₐₓ for cotinine metabolite in liver tissue was 1451±157 ng/g, the tₘₐₓ was 24 hours, the half-life could not be determined, and the exposure for cotinine metabolite based on the dose normalized AUClast was 2505±139 hr*kg*ng/g/mg.

Following PO dosing of Test Nicotine Polacrilex at 10 mg/kg (Groups 5-8), the average (±SE) Cₘₐₓ for nicotine in kidney tissue was 8930±676 ng/g, the tₘₐₓ was 1 hour, the half-life was 24.2 hours, and the exposure for nicotine based on the dose normalized AUClast was 12717±1354 hr*kg*ng/g/mg. After PO dosing of Test Nicotine Polacrilex, the average (±SE) Cₘₐₓ for hydroxycotinine metabolite in kidney tissue was 244±16.5 ng/g, the tₘₐₓ was 24 hours, the half-life could not be determined, and the exposure for hydroxycotinine metabolite based on the dose normalized AUClast was 449±24.1 hr*kg*ng/g/mg. After PO dosing of Test Nicotine Polacrilex, the average (±SE) Cₘₐₓ for nicotine-n-oxide metabolite in kidney tissue was 28.0±6.34 ng/g, the tₘₐₓ was 1 hour, the half-life could not be determined, and the exposure for nicotine-n-oxide metabolite based on the dose normalized AUClast was 38.0±5.57 hr*kg*ng/g/mg. After PO dosing of Test Nicotine Polacrilex, the average (±SE) Cₘₐₓ for cotinine metabolite in kidney tissue was 2466±321 ng/g, the tₘₐₓ was 24 hours, the half-life could not be determined, and the exposure for cotinine metabolite based on the dose normalized AUClast was 4300±280 hr*kg*ng/g/mg.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. Nicotine infused tobacco leaves, comprising:
(a) a therapeutically effective amount of nicotine;
(b) an edible oil; and
(c) tobacco leaves.

2. The nicotine infused tobacco leaves according to claim 1, wherein the edible oil is selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil.

3. The nicotine infused tobacco leaves according to claim 1, wherein the edible oil is sunflower oil.

4. The nicotine infused tobacco leaves according to claim 1, further comprising one or more cannabinoids.

5. The nicotine infused tobacco leaves according to claim 1, further comprising tetrahydrocannabinol, cannabidiol, and mixtures thereof.

6. The nicotine infused tobacco leaves according to claim 1, further comprising a protective colloid chosen from whole milk, skimmed milk, non-fat dry milk or mixtures thereof.

7. The nicotine infused tobacco leaves according to claim 6, wherein the protective colloid is non-fat dry milk.

8. Nicotine infused tobacco leaves, comprising:
(a) a therapeutically effective amount of nicotine;
(b) an edible oil or a protective colloid;
(c) one or more cannabinoids; and
(d) tobacco leaves.

9. The nicotine infused tobacco leaves according to claim 8, comprising an edible oil wherein the edible oil is selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil.

10. The nicotine infused tobacco leaves according to claim 9, wherein the edible oil is sunflower oil.

11. The nicotine infused tobacco leaves according to claim 8, comprising a protective colloid.

12. The nicotine infused tobacco leaves according to claim 11, wherein the protective colloid is non-fat dry milk.

13. The nicotine infused tobacco leaves according to claim 8, wherein the cannabinoid is selected from the group consisting of tetrahydrocannabinol, cannabidiol, and mixtures thereof.

14. Nicotine infused tobacco leaves, comprising:
(a) a therapeutically effective amount of nicotine bound to polymethacrylic acid;
(b) an edible oil; and
(c) tobacco leaves.

15. The nicotine infused tobacco leaves according to claim 14, wherein the edible oil is selected from the group consisting of flaxseed oil, canola oil, coconut oil, corn oil, cottonseed oil, grapeseed oil, peanut oil, olive oil, palm oil, palm kernel oil, rapeseed oil, safflower oil, soybean oil, and sunflower oil.

16. The nicotine infused tobacco leaves according to claim 15, wherein the edible oil is sunflower oil.

17. The nicotine infused tobacco leaves according to claim 14, further comprising one or more cannabinoids.

18. The nicotine infused tobacco leaves according to claim 17, wherein the cannabinoid is selected from the group consisting of tetrahydrocannabinol, cannabidiol, and mixtures thereof.

19. The nicotine infused tobacco leaves according to claim 14, further comprising a binder selected from the group consisting of povidone, sodium carboxymethyl-cellulose and other modified cellulosic materials, sodium alginate, xanthan gum, starch-based binders, gum arabic, pectin, carrageenan, pullulan, and zein.

20. The nicotine infused tobacco leaves according to claim 19, wherein the binder is gum arabic.
